(19) United States
(12) Patent Application Publication
(10) Pub. No.: US 2010/0062424 A1
(43) Pub. Date: Mar. 11, 2010

(54) Assessment of Infectious Bacteria

(75) Inventors: Jim Manos, The University of Sydney (AU); Jonathan Arthur, The University of Sydney (AU); Colin Harbour, The University of Sydney (AU); Barbara Rose, The University of Sydney (AU)

Correspondence Address: SCHWEGMAN, LUNDBERG & WOESSNER, P.A., P.O. BOX 2938, MINNEAPOLIS, MN, 55402, US

(73) Assignee: SYDNEY THE UNIVERSITY OF BUSINESS LIAISON OFFICE, NEW SOUTH WALES (AU)

(21) Appl. No.: 11/992,710

(22) PCT Filed: Sep. 28, 2006
(86) PCT No.: PCT/AU2006/001427

§ 371 Date: Mar. 27, 2008

(30) Foreign Application Priority Data
Sep. 28, 2005 (AU) 2005905356

(51) Int. Cl. C12Q 1/68

(52) U.S. Cl. 435/6

(57) ABSTRACT

Disclosed herein are methods for determining whether an opportunistic bacterium is infectious.

### BACKGROUND OF THE INVENTION

In spite of advances in medicine, bacterial infection continues to be of concern, particularly in compromised individuals. Of particular concern are strains belonging to genus Pseudomonas, including P. aeruginosa, and other pathogens that are capable of forming a bio-film such as B. cenocepacia, E. coli, S. aureus and H. influenzae.

While it has been observed that some strains are more infectious than other strains of the same species, the factors that control characteristics such as transmission of pathogen from individual to individual in many of these strains remain unclear. Accordingly, to date it has been difficult, and in some cases, not possible to identify infectious strains.

There is a need for technology for identifying infectious bacteria and in particular, bacteria that are more or less readily transmissible from individual to individual.

### DESCRIPTION OF THE INVENTION

In certain embodiments there is provided a method for determining whether an opportunistic bacterium is infectious including assessing an opportunistic bacterium for the expression of:
one or more genes shown in Tables 1 to 4; or
one or more genes selected from the group consisting of: PA 0633, PA 0632, PA 0986, PA0637, PA0729, PA1617, PA0639, PA5471, PA5429, PA0636, PA0638, and PA3067; or
one or more genes selected from the group consisting of: PA0053, PA0632, PA0633, PA0634, PA0635, PA0636, PA0637, PA0638, PA0639, PA0730, PA1333, PA1393, PA3528, PA3531, PA3833, PA5429; or
the following genes: PA0632, PA0633, PA0634, PA0635, PA0636, PA0637, PA0638, PA0639; or
gene PA0729; or
the following genes: PA0364, PA1250, PA2587, PA3535, PA3904, PA4590, PA4738, PA4739, PA5220, PA5481, PA1001, PA1177, PA1431, PA1893, PA2007, PA2008, PA2009, PA2513, PA2588, PA3535, PA3904, PA3907, PA3923, PA4206, PA4498, PA4778, PA5061.

TABLE 1
Genes that are absent or that have low relative expression in P1 strains

| Gene | Accession no |
|---|---|
| PA0053 | NP_248743 |
| PA0632 | NP_249323 |
| PA0633 | NP_249324 |
| PA0634 | NP_249325 |
| PA0635 | NP_249326 |
| PA0636 | NP_249327 |
| PA0637 | NP_249328 |
| PA0638 | NP_249329 |
| PA0639 | NP_249330 |
| PA0730 | NP_249421 |
| PA1333 | NP_250024 |
| PA1393 | NP_250084 |
| PA3528 | NP_252218 |
| PA3531 | NP_252221 |
| PA3833 | NP_252522 |
| PA5429 | NP_254116 |

TABLE 2
Genes that have high relative expression in P1 strains

| Gene | Accession no |
|---|---|
| PA0100 | NP_248790 |
| PA0158 | NP_248848 |
| PA0364 | NP_249055 |
| PA0365 | NP_249056 |
| PA0413 | NP_249104 |
| PA0498 | NP_249189 |
| PA0524 | NP_249215 |
| PA0729 | NP_249420 |
| PA0866 | NP_249557 |
| PA0986 | NP_249677 |
| PA0987 | NP_249678 |
| PA1129 | NP_249820 |
| PA1214 | NP_249905 |
| PA1250 | NP_249941 |
| PA1291 | NP_249982 |
| PA1395 | NP_250086 |
| PA1617 | NP_250308 |
| PA1629 | NP_250320 |
| PA1759 | NP_250450 |
| PA1929 | NP_250619 |
| PA2037 | NP_250727 |
| PA2104 | NP_250794 |
| PA2105 | NP_250795 |
| PA2106 | NP_250796 |
| PA2138 | NP_250828 |
| PA2384 | NP_251074 |
| PA2387 | NP_251077 |
| PA2396 | NP_251086 |
| PA2409 | NP_251099 |
| PA2412 | NP_251102 |
| PA2423 | NP_251113 |
| PA2427 | NP_251117 |
| PA2435 | NP_251125 |
| PA2587 | NP_251277 |
| PA2662 | NP_251352 |
| PA2663 | NP_251353 |
| PA3132 | NP_251822 |
| PA3318 | NP_252008 |
| PA3320 | NP_252010 |
| PA3354 | NP_252044 |
| PA3358 | NP_252048 |
| PA3371 | NP_252061 |
| PA3429 | NP_252119 |
| PA3535 | NP_252225 |
| PA3860 | NP_252549 |
| PA3904 | NP_252593 |
| PA3905 | NP_252594 |
| PA4075 | NP_252764 |
| PA4190 | NP_252879 |
| PA4349 | NP_253039 |
| PA4386 | NP_253076 |
| PA4590 | NP_253280 |
| PA4677 | NP_253367 |
| PA4738 | NP_253426 |
| PA4739 | NP_253427 |
| PA4908 | NP_253595 |
| PA4909 | NP_253596 |
| PA4994 | NP_253681 |
| PA5217 | NP_253904 |
| PA5220 | NP_253907 |
| PA5352 | NP_254039 |
| PA5448 | NP_254135 |
| PA5451 | NP_254157 |
| PA5470 | NP_254157 |
| PA5471 | NP_254158 |
| PA5481 | NP_254168 |
| PA5542 | NP_254229 |
| Pae_orfD | |
| Pae_orfH | |
| Pae_orfI | |
| Pae_orfM | |

TABLE 3
Genes that are absent or have low relative expression in P2 strains

| Gene | Accession no |
|---|---|
| PA0053 | NP_248743 |
| PA0092 | NP_248782 |
| PA0978 | NP_249669 |
| PA0979 | NP_249670 |
| PA1333_r_at | NP_250084 |
| PA1471 | NP_250162 |
| PA1571 | NP_250262 |
| PA1581_sdhC | NP_250272 |
| PA1582_sdhD | NP_252273 |
| PA3385 | NP_252075 |
| PA3472 | NP_252162 |
| PA3531_bfrB | NP_252221 |
| PA4431 | NP_253121 |
| PA4563_rpsT_at | NP_253253 |
| PA4568_rpIU | NP_253258 |
| PA4880_at | NP_253567 |
| PA4922_azu | NP_253609 |
| PA5157 | NP_253844 |
| Pae_tRNA_AIa | NP_{—} |

TABLE 4
Genes that have high relative expression in P2 strains

| Gene | Accession no |
|---|---|
| PA0100 | NP_248790 |
| PA0140 | NP_248830 |
| PA0269 | NP_248960 |
| PA0270 | NP_248961 |
| PA0271 | NP_248962 |
| PA0852 | NP_249563 |
| PA0986 | NP_249677 |
| PA1250 | NP_249941 |
| PA1871 | NP_250562 |
| PA2034 | NP_250724 |
| PA2037 | NP_250727 |
| PA2069 | NP_250759 |
| PA2104 | NP_250794 |
| PA2105 | NP_250795 |
| PA2300 | NP_250990 |
| PA2303 | NP_250993 |
| PA2304 | NP_250994 |
| PA2305 | NP_250995 |
| PA2384 | NP_251074 |
| PA2387 | NP_251077 |
| PA2396 | NP_251086 |
| PA2398 | NP_251088 |
| PA2400 | NP_251090 |
| PA2402 | NP_251092 |
| PA2411 | NP_251101 |
| PA2412 | NP_251102 |
| PA2456 | NP_251146 |
| PA2476 | NP_251166 |
| PA2564 | NP_251254 |
| PA2565 | NP_251565 |
| PA2587 | NP_251277 |
| PA2588 | NP_251278 |
| PA3477 | NP_252167 |
| PA3478 | NP_252168 |
| PA3479 | NP_252169 |
| PA3520 | NP_252210 |
| PA3577 | NP_252267 |
| PA3810 | NP_252499 |
| PA3811 | NP_252500 |
| PA3812 | NP_252501 |
| PA3813 | NP_252502 |
| PA3867 | NP_252556 |
| PA4078 | NP_252767 |
| PA4079 | NP_252768 |
| PA4141 | NP_252830 |
| PA4205 | NP_252894 |
| PA4206 | NP_252895 |
| PA4207 | NP_252896 |
| PA4208 | NP_252897 |
| PA4471 | NP_253161 |
| Pae_L37109cds1 | NP_{—} |
| Pae_L37109cds2 | NP_{—} |

These genes and those shown in Tables 1 to 4 have been identified in the genome of P. aeruginosa strain PA01 (Stover C at al. 2000 Nature 406: 959). The sequence of these genes is publicly available at: http://www.pseudomonas.com/BrowseByFunctionU.asp

It will be understood that an opportunistic bacterium is a bacterium that tends to cause disease in compromised individuals. Individuals having one or more mutations leading to a detrimental genetic abnormality, for example, an individual with cystic fibrosis, is an example of a compromised individual. Other compromised individuals include those having: compromised immunity, such as immuno-deficient or immuno-suppressed individuals, for example, individuals with AIDS; compromised anatomy, such as injury, especially burns; compromised metabolism, for example, due to drug treatment. Those having stress or poor diet are also examples of compromised individuals.

Further, it will be understood that particular examples of opportunistic bacteria include bacteria that tend to produce a bio-film during certain phases of infection. A bio-film is a composition secreted by a bacteria that consists generally of polysaccharides and glycoproteins and of bacteria themselves. Bacteria that produce bio-films are of particular concern as they are less readily treated by antibiotictherapy. Examples include bacteria of genus Burkholderia such as B. cenocepacia.

In certain embodiments, it will be understood that an opportunistic bacteria is a Pseudomonad, or in other words, a bacteria of genus Pseudomonas. Pseudomonas aeruginosa (P. aeruginosa) is of particular interest because it tends to be found in many different environments and is able to vary its genetic composition and while many strains have low infectivity, even in a compromised individual, others discussed further herein are highly infectious. For example, it has been observed from studies of various populations of individuals having cystic fibrosis and being infected with P. aeruginosa that some strains appear to be readily transmitted between individuals, or otherwise are readily taken up from an environment, whereas other strains are not.

In summary of the above, it will be understood that an opportunistic bacterium is typically a bacterium that is capable of infecting compromised individuals, that is capable of forming a bio-film at some point during infection and that is generally a Pseudomonad or like bacteria.

As discussed herein, it has been observed from studies of various populations of individuals infected with a species of opportunistic bacteria that a greater proportion of individuals in any given population tend to contain a particular strain. For example, as discussed herein, an increasing proportion of individuals having cystic fibrosis and grouped according to age tend to be infected with a particular strain of P. aeruginosa rather than other strains of this species.

It will be understood that strains that tend to dominate, or otherwise that are highly represented in a population are infectious. These strains are also known as transmissible, contagious or communicable strains.

A strain that is infectious may be one that is more or less readily transmitted from one individual to another, whether by direct contact or otherwise. Alternatively a strain that is infectious may be one that is more or less readily taken up from another environment, such as a clinic, again, by direct contact or otherwise.

Examples of infectious strains of P. aeruginosa include pulsotype I (“P1”) discussed in Anthony M. et al. J Clin Microbiol, 2002. 40(8):p. 2772-8; LES400 and LES 431 discussed in Salunkhe P. et al. J Bacteriol, 2005. 187(14):p. 4908-20 and MA discussed in Lewis D A et al. Am J Respir Cell Mol Biol, 2005. 33(1):p. 56-64. These strains tend to be found in greater numbers in individuals having cystic fibrosis than do other P. aeruginosa strains.

As discussed below, the inventors have compared infectious and non infectious strains of bacteria of the same species and from this analysis have identified genetic markers that are characteristic of infectious bacteria strains and that can be used to distinguish infectious and non infectious strains of bacteria of the same species.

More particularly, the inventors have found that the level of expression of certain bacterial genes is characteristic of a capacity of a strain to infect an individual. In certain embodiments, a relatively low level of gene expression, (as compared with the level of expression of the same gene in a non infectious strain), or otherwise no expression, is characteristic of an infectious strain. In other embodiment, a relatively high level of gene expression is characteristic of an infectious strain.

According to the methods described herein, the level of gene expression may be assessed by any method that is capable of measuring gene expression. For example the amount of transcribed mRNA or gene product may be measured. Transcribed mRNA may be measured by quantitative PCR, micro-array technologies or simple Northern blotting with or without densitometry. Examples of PCR and micro-array approaches are discussed further herein. Gene products, such as peptides and polypeptides may be measured by any serological approach including ELISA, Western blotting, protein micro-array etc.

In certain embodiments, gene expression is assessed by determining whether the nucleotide sequence of the subject gene has one or more mutations such as substitution, insertion or deletion. In other embodiments, gene expression is assessed by determining whether a subject gene has been partially or wholly deleted or otherwise lost from the genome of a strain. In other embodiments, gene expression is assessed by determining whether an operon has been partially or wholly deleted or otherwise lost from the genome of a strain.

In other embodiments, gene expression is assessed by determining the inclusion of a gene.

In certain embodiments the method discussed above includes the further step of assessing the opportunistic bacterium for the expression of one or more genes shown in Table 5.

TABLE 5

| Function class | Genes |
|---|---|
| Adaptation, | PA0031 PA0059 PA0074 PA0139 PA0140 PA0173 PA0175 PA0176 |
| protection (for | PA0177 PA0178 PA0179 PA0180 PA0456 PA0594 PA0595 PA0848 |
| example cold | PA0923 PA0934 PA0950 PA0962 PA1001 PA1002 PA1008 PA1127 |
| shock proteins) | PA1130 PA1150 PA1151 PA1159 PA1178 PA1251 PA1338 PA1423 |
| | PA1430 PA1431 PA1432 PA1443 PA1444 PA1445 PA1446 PA1447 |
| | PA1448 PA1449 PA1452 PA1453 PA1457 PA1464 PA1561 PA1608 |
| | PA1646 PA1782 PA1803 PA1813 PA1930 PA1959 PA2064 PA2065 |
| | PA2147 PA2185 PA2279 PA2385 PA2386 PA2392 PA2394 PA2395 |
| | PA2396 PA2397 PA2399 PA2400 PA2411 PA2413 PA2424 PA2425 |
| | PA2475 PA2532 PA2561 PA2570 PA2573 PA2622 PA2652 PA2654 |
| | PA2788 PA2826 PA2830 PA2832 PA2850 PA2867 PA2920 PA2978 |
| | PA3007 PA3109 PA3236 PA3266 PA3327 PA3331 PA3348 PA3349 |
| | PA3450 PA3475 PA3476 PA3477 PA3529 PA3531 PA3540 PA3541 |
| | PA3542 PA3543 PA3544 PA3545 PA3546 PA3547 PA3548 PA3549 |
| | PA3550 PA3551 PA3553 PA3573 PA3624 PA3625 PA3706 PA3708 |
| | PA3818 PA3866 PA4078 PA4110 PA4235 PA4236 PA4290 PA4307 |
| | PA4309 PA4310 PA4356 PA4366 PA4378 PA4427 PA4428 PA4446 |
| | PA4468 PA4472 PA4520 PA4557 PA4566 PA4606 PA4612 PA4613 |
| | PA4614 PA4633 PA4671 PA4723 PA4743 PA4760 PA4761 PA4827 |
| | PA4844 PA4876 PA4915 PA4945 PA4953 PA4954 PA5072 PA5117 |
| | PA5160 PA5241 PA5242 PA5272 PA5338 PA5372 PA5373 |
| Amino acid | PA0008 PA0009 PA0018 PA0025 PA0035 PA0036 PA0037 PA0132 |
| biosynthesis and | PA0164 PA0223 PA0245 PA0265 PA0266 PA0288 PA0292 PA0293 |
| metabolism | PA0297 PA0316 PA0331 PA0353 PA0361 PA0390 PA0393 PA0399 |
| | PA0400 PA0402 PA0407 PA0430 PA0432 PA0447 PA0546 PA0609 |
| | PA0649 PA0650 PA0651 PA0662 PA0668 PA0761 PA0782 PA0826.1 |
| | PA0826.2 PA0865 PA0870 PA0871 PA0872 PA0893 PA0895 PA0896 |
| | PA0897 PA0898 PA0899 PA0901 PA0904 PA0932 PA0956 PA1002 |
| | PA1004 PA1010 PA1162 PA1217 PA1326 PA1337 PA1338 PA1375 |
| | PA1393 PA1421 PA1585 PA1587 PA1681 PA1684 PA1685 PA1687 |
| | PA1750 PA1754 PA1756 PA1757 PA1794 PA1795 PA1812 PA1818 |
| | PA1843 PA1927 PA1999 PA2000 PA2025 PA2084 PA2104 PA2247 |
| | PA2248 PA2249 PA2250 PA2253 PA2442 PA2443 PA2444 PA2445 |
| | PA2446 PA2531 PA2612 PA2623 PA2629 PA2683 PA2709 PA2744 |
| | PA2923 PA2924 PA2925 PA2926 PA2943 PA3013 PA3014 PA3068 |
| | PA3082 PA3107 PA3108 PA3113 PA3116 PA3117 PA3118 PA3120 |
| | PA3121 PA3139 PA3151 PA3152 PA3164 PA3165 PA3166 PA3167 |
| | PA3175 PA3357 PA3418 PA3459 PA3475 PA3482 PA3525 PA3537 |
| | PA3570 PA3736 PA3751 PA3769 PA3792 PA3814 PA3816 PA3834 |
| | PA3987 PA3997 PA4007 PA4138 PA4180 PA4402 PA4439 PA4442 |
| | PA4443 PA4447 PA4448 PA4449 PA4519 PA4548 PA4560 PA4565 |
| | PA4588 PA4602 PA4666 PA4694 PA4695 PA4696 PA4731 PA4756 |
| | PA4758 PA4759 PA4839 PA4846 PA4920 PA4930 PA4938 PA4957 |
| | PA4960 PA4976 PA4977 PA5013 PA5015 PA5025 PA5035 PA5036 |
| | PA5038 PA5039 PA5066 PA5067 PA5091 PA5092 PA5098 PA5100 |
| | PA5119 PA5140 PA5141 PA5142 PA5143 PA5170 PA5171 PA5172 |
| | PA5173 PA5203 PA5204 PA5206 PA5213 PA5214 PA5215 PA5263 |
| | PA5277 PA5278 PA5302 PA5304 PA5322 PA5323 PA5372 PA5373 |
| | PA5379 PA5413 PA5415 PA5429 PA5495 |
| Antibiotic | PA0425 PA0426 PA0427 PA0706 PA1032 PA1129 PA1131 PA1858 |
| resistance and | PA1959 PA2018 PA2019 PA2055 PA2493 PA2494 PA2495 PA3556 |
| susceptibility | PA4119 PA4356 PA4597 PA4598 PA4599 PA4777 PA5514 |
| Biosynthesis of | PA0024 PA0342 PA0350 PA0353 PA0381 PA0407 PA0420 PA0500 |
| cofactors, | PA0501 PA0502 PA0503 PA0504 PA0509 PA0510 PA0511 PA0512 |
| prosthetic groups | PA0513 PA0514 PA0515 PA0516 PA0517 PA0518 PA0551 PA0582 |
| and carriers | PA0583 PA0593 PA0649 PA0672 PA0761 PA0773 PA0996 PA0997 |
| | PA0998 PA0999 PA1000 PA1003 PA1004 PA1017 PA1049 PA1125 |
| | PA1272 PA1273 PA1275 PA1276 PA1277 PA1278 PA1279 PA1280 |
| | PA1281 PA1375 PA1505 PA1546 PA1674 PA1758 PA1772 PA1778 |
| | PA1796 PA1923 PA1973 PA1985 PA1986 PA1987 PA1988 PA1989 |
| | PA2025 PA2103 PA2587 PA2611 PA2666 PA2903 PA2904 PA2905 |
| | PA2907 PA2908 PA2909 PA2942 PA2944 PA2945 PA2947 PA2948 |
| | PA2964 PA3028 PA3029 PA3030 PA3111 PA3167 PA3171 PA3397 |
| | PA3438 PA3439 PA3627 PA3633 PA3650 PA3652 PA3735 PA3812 |
| | PA3813 PA3814 PA3816 PA3870 PA3914 PA3915 PA3916 PA3917 |
| | PA3918 PA3975 PA3976 PA3977 PA3996 PA3997 PA4006 PA4007 |
| | PA4043 PA4044 PA4047 PA4051 PA4053 PA4054 PA4055 PA4056 |
| | PA4280 PA4376 PA4397 PA4524 PA4529 PA4561 PA4569 PA4655 |
| | PA4663 PA4664 PA4666 PA4669 PA4694 PA4695 PA4696 PA4728 |
| | PA4729 PA4730 PA4731 PA4750 PA4891 PA4892 PA4893 PA4919 |
| | PA4920 PA4973 PA5034 PA5063 PA5065 PA5118 PA5203 PA5223 |
| | PA5243 PA5259 PA5260 PA5320 PA5357 PA5358 PA5516 |
| Carbon compound | PA0152 PA0153 PA0154 PA0155 PA0183 PA0208 PA0210 PA0211 |
| catabolism | PA0212 PA0213 PA0214 PA0226 PA0227 PA0228 PA0230 PA0231 |
| | PA0232 PA0235 PA0247 PA0265 PA0266 PA0447 PA0482 PA0552 |
| | PA0555 PA0608 PA0743 PA0792 PA0795 PA0796 PA0810 PA0835 |
| | PA0854 PA0887 PA0927 PA1020 PA1146 PA1310 PA1311 PA1375 |
| | PA1384 PA1409 PA1498 PA1515 PA1525 PA1602 PA1629 PA1726 |
| | PA1770 PA1931 PA1932 PA1933 PA1949 PA1950 PA1982 PA1983 |
| | PA1999 PA2000 PA2003 PA2007 PA2008 PA2009 PA2011 PA2012 |
| | PA2013 PA2014 PA2015 PA2083 PA2085 PA2086 PA2097 PA2098 |
| | PA2260 PA2261 PA2265 PA2266 PA2290 PA2300 PA2321 PA2323 |
| | PA2342 PA2343 PA2344 PA2356 PA2357 PA2414 PA2416 PA2470 |
| | PA2507 PA2508 PA2509 PA2510 PA2512 PA2513 PA2514 PA2515 |
| | PA2516 PA2517 PA2518 PA2574 PA2623 PA2796 PA2862 PA2932 |
| | PA3025 PA3131 PA3181 PA3183 PA3184 PA3192 PA3193 PA3194 |
| | PA3195 PA3331 PA3363 PA3366 PA3517 PA3560 PA3563 PA3569 |
| | PA3570 PA3588 PA3589 PA3590 PA3591 PA3635 PA3636 PA3710 |
| | PA3935 PA4025 PA4091 PA4092 PA4123 PA4124 PA4125 PA4126 |
| | PA4127 PA4150 PA4151 PA4152 PA4153 PA4329 PA4412 PA4590 |
| | PA4670 PA4725 PA4726 PA4732 PA4733 PA4901 PA4904 PA4905 |
| | PA5016 PA5057 PA5110 PA5131 PA5161 PA5192 PA5332 PA5349 |
| | PA5350 PA5351 PA5353 PA5354 PA5355 PA5410 PA5411 PA5416 |
| | PA5417 PA5418 PA5419 PA5427 |
| Cell wall, LPS | PA0011 PA0223 PA0378 PA0395 PA0763 PA0764 PA0765 PA0766 |
| | PA0869 PA0936 PA1077 PA1078 PA1079 PA1080 PA1081 PA1082 |
| | PA1083 PA1084 PA1085 PA1086 PA1087 PA1094 PA1100 PA1101 |
| | PA1102 PA1103 PA1105 PA1130 PA1171 PA1222 PA1385 PA1812 |
| | PA1959 PA2144 PA2232 PA2234 PA2272 PA2977 PA2978 PA2979 |
| | PA2981 PA3020 PA3047 PA3141 PA3145 PA3146 PA3147 PA3148 |
| | PA3149 PA3150 PA3153 PA3154 PA3155 PA3156 PA3157 PA3158 |
| | PA3159 PA3160 PA3242 PA3337 PA3387 PA3540 PA3541 PA3542 |
| | PA3543 PA3545 PA3546 PA3547 PA3548 PA3549 PA3550 PA3551 |
| | PA3556 PA3636 PA3643 PA3644 PA3645 PA3646 PA3652 PA3666 |
| | PA3984 PA3999 PA4001 PA4002 PA4003 PA4020 PA4201 PA4406 |
| | PA4410 PA4411 PA4412 PA4414 PA4415 PA4416 PA4417 PA4418 |
| | PA4444 PA4450 PA4479 PA4480 PA4481 PA4512 PA4522 PA4545 |
| | PA4662 PA4668 PA4700 PA4749 PA4776 PA4947 PA4988 PA4996 |
| | PA4997 PA5009 PA5010 PA5011 PA5012 PA5045 PA5077 PA5161 |
| | PA5162 PA5163 PA5164 PA5238 PA5276 PA5322 PA5447 PA5448 |
| | PA5449 PA5450 PA5451 PA5452 PA5453 PA5454 PA5538 PA5552 |
| Central | PA0102 PA0183 PA0265 PA0266 PA0363 PA0430 PA0482 PA0520 |
| intermediary | PA0546 PA0555 PA0654 PA0710 PA0795 PA0796 PA0887 PA0927 |
| metabolism | PA1338 PA1376 PA1384 PA1393 PA1502 PA1614 PA1770 PA1779 |
| | PA1780 PA1781 PA1838 PA2001 PA2023 PA2052 PA2053 PA2140 |
| | PA2193 PA2194 PA2195 PA2357 PA2393 PA2442 PA2445 PA2473 |
| | PA2717 PA2726 PA2787 PA2813 PA2821 PA2990 PA3131 PA3181 |
| | PA3182 PA3296 PA3452 PA3471 PA3524 PA3561 PA3562 PA3582 |
| | PA3584 PA3629 PA3687 PA3758 PA4024 PA4031 PA4068 PA4100 |
| | PA4130 PA4442 PA4443 PA4563 PA4626 PA4640 PA4733 PA4748 |
| | PA4864 PA4865 PA4867 PA4868 PA4880 PA4956 PA5046 PA5056 |
| | PA5058 PA5060 PA5110 PA5111 PA5213 PA5214 PA5215 PA5288 |
| | PA5297 PA5308 PA5353 PA5354 PA5355 PA5421 PA5435 PA5436 |
| | PA5549 PA5570 |
| Chaperones & | PA0499 PA0538 PA0594 PA0699 PA0837 PA1068 PA1481 PA1596 |
| heat shock | PA1793 PA1800 PA1801 PA1802 PA1805 PA1996 PA2129 PA2132 |
| proteins | PA2476 PA2614 PA2725 PA3126 PA3221 PA3227 PA3262 PA3365 |
| | PA3717 PA3737 PA3810 PA3811 PA3842 PA3871 PA4083 PA4085 |
| | PA4176 PA4385 PA4386 PA4558 PA4572 PA4651 PA4760 PA4761 |
| | PA4762 PA4845 PA4873 PA5053 PA5054 PA5193 PA5195 PA5254 |
| | PA5256 PA5459 |
| Chemotaxis | PA0173 PA0175 PA0176 PA0177 PA0178 PA0179 PA0180 PA0408 |
| | PA0409 PA0410 PA0411 PA0412 PA0413 PA0414 PA0415 PA0416 |
| | PA0417 PA1251 PA1423 PA1443 PA1444 PA1445 PA1446 PA1447 |
| | PA1448 PA1449 PA1452 PA1453 PA1456 PA1457 PA1458 PA1459 |
| | PA1460 PA1461 PA1464 PA1561 PA1608 PA1646 PA1930 PA2561 |
| | PA2573 PA2652 PA2654 PA2788 PA2867 PA2920 PA3348 PA3349 |
| | PA3702 PA3703 PA3704 PA3705 PA3706 PA3707 PA3708 PA4290 |
| | PA4307 PA4309 PA4310 PA4520 PA4633 PA4844 PA4915 PA4953 |
| | PA4954 PA5072 |
| DNA replication, | PA0001 PA0002 PA0003 PA0004 PA0010 PA0357 PA0382 PA0577 |
| recombination, | PA0669 PA0720 PA0750 PA0772 PA0923 PA0965 PA0966 PA0967 |
| modification and | PA0995 PA1405 PA1529 PA1532 PA1534 PA1686 PA1804 PA1815 |
| repair | PA1886 PA2118 PA2138 PA2545 PA2585 PA2738 PA2749 PA2961 |
| | PA3002 PA3011 PA3138 PA3161 PA3168 PA3272 PA3344 PA3495 |
| | PA3528 PA3596 PA3617 PA3620 PA3640 PA3642 PA3725 PA3738 |
| | PA3745 PA3777 PA3832 PA3867 PA3940 PA3989 PA4042 PA4172 |
| | PA4232 PA4234 PA4281 PA4282 PA4283 PA4284 PA4285 PA4316 |
| | PA4363 PA4400 PA4609 PA4660 PA4760 PA4761 PA4762 PA4763 |
| | PA4853 PA4931 PA4946 PA4964 PA4967 PA5050 PA5147 PA5280 |
| | PA5296 PA5319 PA5345 PA5348 PA5443 PA5493 |
| Energy | PA0023 PA0105 PA0106 PA0107 PA0108 PA0113 PA0195 PA0196 |
| metabolism | PA0330 PA0362 PA0509 PA0510 PA0511 PA0512 PA0513 PA0514 |
| | PA0515 PA0516 PA0517 PA0518 PA0519 PA0520 PA0321 PA0523 |
| | PA0524 PA0525 PA0548 PA0552 PA0589 PA0607 PA0609 PA0649 |
| | PA0794 PA0854 PA0918 PA0927 PA1104 PA1172 PA1173 PA1174 |
| | PA1175 PA1176 PA1177 PA1317 PA1318 PA1319 PA1320 PA1321 |
| | PA1400 PA1479 PA1480 PA1481 PA1482 PA1483 PA1498 PA1551 |
| | PA1552 PA1553 PA1554 PA1555 PA1556 PA1557 PA1562 PA1580 |
| | PA1581 PA1582 PA1583 PA1584 PA1585 PA1586 PA1587 PA1588 |
| | PA1589 PA1600 PA1770 PA1787 PA1883 PA1931 PA1983 PA2153 |
| | PA2165 PA2250 PA2266 PA2290 PA2297 PA2321 PA2382 PA2482 |
| | PA2516 PA2623 PA2624 PA2637 PA2638 PA2639 PA2640 PA2641 |
| | PA2642 PA2643 PA2644 PA2645 PA2646 PA2647 PA2648 PA2649 |
| | PA2664 PA2680 PA2694 PA2714 PA2715 PA2716 PA2796 PA2951 |
| | PA2952 PA2953 PA2994 PA2995 PA2996 PA2997 PA2998 PA2999 |
| | PA3024 PA3025 PA3032 PA3171 PA3183 PA3193 PA3194 PA3195 |
| | PA3391 PA3392 PA3393 PA3394 PA3395 PA3396 PA3397 PA3415 |
| | PA3416 PA3417 PA3452 PA3490 PA3491 PA3584 PA3621 PA3635 |
| | PA3687 PA3809 PA3872 PA3873 PA3874 PA3875 PA3878 PA3879 |
| | PA3929 PA3930 PA4061 PA4133 PA4329 PA4331 PA4333 PA4429 |
| | PA4430 PA4470 PA4538 PA4569 PA4571 PA4587 PA4640 PA4732 |
| | PA4748 PA4771 PA4772 PA4809 PA4810 PA4811 PA4812 PA4829 |
| | PA4922 PA4971 PA4975 PA5015 PA5016 PA5063 PA5129 PA5192 |
| | PA5223 PA5240 PA5297 PA5300 PA5304 PA5328 PA5332 PA5358 |
| | PA5399 PA5400 PA5427 PA5439 PA5490 PA5491 PA5553 PA5554 |
| | PA5555 PA5556 PA5557 PA5558 PA5559 PA5560 PA5561 |
| Membrane | PA0007 PA0013 PA0021 PA0029 PA0043 PA0070 PA0072 PA0103 |
| proteins | PA0109 PA0112 PA0137 PA0138 PA0150 PA0158 PA0162 PA0166 |
| | PA0185 PA0188 PA0189 PA0192 PA0204 PA0205 PA0215 PA0216 |
| | PA0229 PA0235 PA0239 PA0240 PA0241 PA0246 PA0260 PA0273 |
| | PA0276 PA0281 PA0282 PA0285 PA0291 PA0303 PA0304 PA0313 |
| | PA0322 PA0324 PA0325 PA0334 PA0340 PA0345 PA0349 PA0352 |
| | PA0365 PA0426 PA0427 PA0434 PA0443 PA0450 PA0451 PA0452 |
| | PA0458 PA0465 PA0471 PA0485 PA0539 PA0567 PA0575 PA0605 |
| | PA0606 PA0659 PA0661 PA0676 PA0693 PA0694 PA0702 PA0703 |
| | PA0736 PA0738 PA0751 PA0752 PA0753 PA0755 PA0783 PA0786 |
| | PA0789 PA0790 PA0800 PA0801 PA0802 PA0806 PA0809 PA0811 |
| | PA0833 PA0847 PA0860 PA0861 PA0889 PA0890 PA0909 PA0920 |
| | PA0921 PA0924 PA0969 PA0971 PA0973 PA0984 PA0985 PA1007 |
| | PA1019 PA1025 PA1041 PA1042 PA1044 PA1048 PA1051 PA1053 |
| | PA1054 PA1055 PA1056 PA1057 PA1058 PA1059 PA1060 PA1072 |
| | PA1073 PA1107 PA1108 PA1113 PA1115 PA1118 PA1119 PA1120 |
| | PA1126 PA1131 PA1144 PA1147 PA1170 PA1178 PA1181 PA1190 |
| | PA1194 PA1199 PA1209 PA1212 PA1230 PA1231 PA1232 PA1236 |
| | PA1245 PA1257 PA1258 PA1262 PA1265 PA1282 PA1286 PA1288 |
| | PA1297 PA1301 PA1302 PA1305 PA1308 PA1313 PA1316 PA1322 |
| | PA1341 PA1352 PA1360 PA1361 PA1364 PA1408 PA1411 PA1419 |
| | PA1429 PA1433 PA1436 PA1442 PA1476 PA1477 PA1485 PA1491 |
| | PA1496 PA1497 PA1507 PA1513 PA1519 PA1540 PA1541 PA1547 |
| | PA1549 PA1569 PA1577 PA1591 PA1595 PA1613 PA1620 PA1625 |
| | PA1626 PA1647 PA1650 PA1651 PA1652 PA1669 PA1676 PA1682 |
| | PA1698 PA1727 PA1735 PA1767 PA1773 PA1775 PA1777 PA1783 |
| | PA1819 PA1820 PA1824 PA1825 PA1848 PA1851 PA1857 PA1873 |
| | PA1882 PA1891 PA1895 PA1897 PA1908 PA1911 PA1958 PA1960 |
| | PA1972 PA1977 PA1993 PA2002 PA2004 PA2006 PA2018 PA2026 |
| | PA2039 PA2051 PA2055 PA2059 PA2068 PA2070 PA2072 PA2091 |
| | PA2092 PA2094 PA2101 PA2114 PA2135 PA2148 PA2154 PA2200 |
| | PA2202 PA2203 PA2210 PA2213 PA2214 PA2219 PA2241 PA2262 |
| | PA2269 PA2278 PA2286 PA2295 PA2306 PA2307 PA2313 PA2314 |
| | PA2327 PA2331 PA2351 PA2369 PA2375 PA2388 PA2390 PA2391 |
| | PA2397 PA2403 PA2404 PA2409 PA2415 PA2420 PA2431 PA2435 |
| | PA2437 PA2439 PA2465 PA2467 PA2472 PA2477 PA2478 PA2494 |
| | PA2495 PA2500 PA2501 PA2520 PA2521 PA2522 PA2525 PA2526 |
| | PA2527 PA2533 PA2538 PA2539 PA2549 PA2558 PA2563 PA2568 |
| | PA2576 PA2589 PA2628 PA2653 PA2662 PA2663 PA2678 PA2688 |
| | PA2697 PA2700 PA2701 PA2710 PA2712 PA2746 PA2751 PA2752 |
| | PA2757 PA2761 PA2763 PA2768 PA2773 PA2774 PA2775 PA2777 |
| | PA2784 PA2811 PA2818 PA2835 PA2853 PA2858 PA2868 PA2870 |
| | PA2873 PA2884 PA2900 PA2910 PA2911 PA2914 PA2916 PA2919 |
| | PA2924 PA2925 PA2929 PA2933 PA2936 PA2938 PA2946 PA2984 |
| | PA2985 PA2988 PA3039 PA3041 PA3058 PA3060 PA3065 PA3079 |
| | PA3086 PA3115 PA3137 PA3141 PA3145 PA3153 PA3154 PA3201 |
| | PA3210 PA3211 PA3216 PA3222 PA3228 PA3231 PA3234 PA3235 |
| | PA3252 PA3253 PA3264 PA3265 PA3267 PA3268 PA3275 PA3278 |
| | PA3281 PA3303 PA3305 PA3310 PA3311 PA3315 PA3316 PA3320 |
| | PA3336 PA3339 PA3340 PA3342 PA3343 PA3355 PA3358 PA3359 |
| | PA3362 PA3369 PA3370 PA3382 PA3391 PA3395 PA3400 PA3401 |
| | PA3403 PA3409 PA3428 PA3431 PA3432 PA3443 PA3448 PA3457 |
| | PA3465 PA3467 PA3473 PA3474 PA3489 PA3491 PA3512 PA3521 |
| | PA3522 PA3526 PA3532 PA3544 PA3556 PA3557 PA3558 PA3575 |
| | PA3585 PA3588 PA3595 PA3597 PA3602 PA3605 PA3608 PA3609 |
| | PA3647 PA3648 PA3660 PA3665 PA3671 PA3676 PA3690 PA3692 |
| | PA3709 PA3718 PA3730 PA3739 PA3747 PA3748 PA3749 PA3773 |
| | PA3775 PA3780 PA3781 PA3788 PA3789 PA3791 PA3794 PA3819 |
| | PA3821 PA3826 PA3827 PA3828 PA3837 PA3839 PA3876 PA3877 |
| | PA3887 PA3888 PA3890 PA3893 PA3894 PA3897 PA3900 PA3901 |
| | PA3920 PA3926 PA3933 PA3934 PA3936 PA3955 PA3963 PA3966 |
| | PA4008 PA4011 PA4013 PA4014 PA4016 PA4034 PA4038 PA4046 |
| | PA4065 PA4067 PA4096 PA4113 PA4126 PA4136 PA4143 PA4160 |
| | PA4161 PA4179 PA4187 PA4193 PA4194 PA4205 PA4207 PA4208 |
| | PA4218 PA4219 PA4223 PA4224 PA4233 PA4243 PA4287 PA4289 |
| | PA4292 PA4297 PA4300 PA4301 PA4317 PA4318 PA4319 PA4320 |
| | PA4332 PA4334 PA4338 PA4343 PA4355 PA4358 PA4365 PA4367 |
| | PA4370 PA4375 PA4383 PA4387 PA4455 PA4467 PA4503 PA4504 |
| | PA4507 PA4521 PA4532 PA4541 PA4586 PA4589 PA4593 PA4597 |
| | PA4598 PA4601 PA4614 PA4622 PA4628 PA4647 PA4654 PA4688 |
| | PA4690 PA4707 PA4719 PA4747 PA4754 PA4757 PA4765 PA4779 |
| | PA4783 PA4804 PA4815 PA4818 PA4821 PA4822 PA4833 PA4834 |
| | PA4835 PA4837 PA4849 PA4857 PA4859 PA4860 PA4876 PA4887 |
| | PA4894 PA4895 PA4898 PA4900 PA4903 PA4911 PA4912 PA4924 |
| | PA4929 PA4933 PA4940 PA4953 PA4961 PA4962 PA4981 PA4990 |
| | PA4999 PA5002 PA5021 PA5030 PA5070 PA5075 PA5095 PA5097 |
| | PA5099 PA5102 PA5107 PA5113 PA5114 PA5121 PA5132 PA5133 |
| | PA5151 PA5154 PA5155 PA5160 PA5167 PA5168 PA5169 PA5170 |
| | PA5182 PA5183 PA5194 PA5205 PA5207 PA5211 PA5216 PA5219 |
| | PA5230 PA5231 PA5235 PA5238 PA5244 PA5248 PA5249 PA5250 |
| | PA5251 PA5264 PA5265 PA5268 PA5282 PA5287 PA5291 PA5311 |
| | PA5365 PA5366 PA5367 PA5368 PA5370 PA5375 PA5377 PA5409 |
| | PA5424 PA5430 PA5434 PA5442 PA5444 PA5451 PA5458 PA5466 |
| | PA5469 PA5473 PA5476 PA5477 PA5478 PA5479 PA5482 PA5501 |
| | PA5504 PA5505 PA5510 PA5515 PA5518 PA5528 PA5529 PA5530 |
| | PA5544 PA5548 PA5561 PA5568 |
| Motility & | PA0171 PA0395 PA0396 PA0408 PA0409 PA0410 PA0411 PA0412 |
| attachment | PA0413 PA0499 PA0992 PA0993 PA0994 PA1077 PA1078 PA1079 |
| | PA1080 PA1081 PA1082 PA1083 PA1084 PA1085 PA1086 PA1087 |
| | PA1092 PA1094 PA1097 PA1099 PA1100 PA1101 PA1102 PA1103 |
| | PA1104 PA1105 PA1443 PA1444 PA1445 PA1446 PA1447 PA1448 |
| | PA1449 PA1452 PA1453 PA1454 PA1460 PA1461 PA1822 PA2126 |
| | PA2129 PA2130 PA2131 PA2132 PA2407 PA2570 PA2960 PA3115 |
| | PA3361 PA3702 PA3703 PA3704 PA3705 PA3706 PA3707 PA3708 |
| | PA3805 PA4081 PA4082 PA4083 PA4084 PA4085 PA4086 PA4525 |
| | PA4526 PA4527 PA4528 PA4546 PA4547 PA4549 PA4550 PA4551 |
| | PA4552 PA4553 PA4554 PA4555 PA4556 PA4651 PA4953 PA4959 |
| | PA5040 PA5041 PA5042 PA5043 PA5044 PA5498 |
| Nucleotide | PA0134 PA0143 PA0148 PA0336 PA0342 PA0401 PA0402 PA0403 |
| biosynthesis and | PA0437 PA0438 PA0441 PA0444 PA0590 PA0849 PA0944 PA0945 |
| metabolism | PA1013 PA1124 PA1155 PA1156 PA1384 PA1393 PA1521 PA1523 |
| | PA1524 PA1543 PA1796 PA1816 PA2616 PA2629 PA2832 PA2876 |
| | PA2962 PA2991 PA3004 PA3050 PA3108 PA3163 PA3480 PA3527 |
| | PA3637 PA3654 PA3686 PA3751 PA3763 PA3769 PA3770 PA3807 |
| | PA3970 PA4314 PA4397 PA4645 PA4646 PA4670 PA4756 PA4758 |
| | PA4854 PA4855 PA4938 PA5129 PA5240 PA5241 PA5242 PA5298 |
| | PA5321 PA5331 PA5336 PA5338 PA5420 PA5425 PA5426 PA3541 |
| Putative enzymes | PA0051 PA0075 PA0117 PA0118 PA0130 PA0142 PA0147 PA0182 |
| | PA0190 PA0193 PA0194 PA0202 PA0209 PA0219 PA0221 PA0224 |
| | PA0237 PA0242 PA0244 PA0249 PA0296 PA0298 PA0299 PA0318 |
| | PA0321 PA0333 PA0364 PA0366 PA0372 PA0386 PA0421 PA0439 |
| | PA0440 PA0473 PA0478 PA0480 PA0483 PA0489 PA0493 PA0494 |
| | PA0506 PA0507 PA0508 PA0530 PA0531 PA0534 PA0545 PA0562 |
| | PA0597 PA0656 PA0657 PA0658 PA0669 PA0704 PA0705 PA0728 |
| | PA0730 PA0740 PA0744 PA0745 PA0746 PA0747 PA0766 PA0779 |
| | PA0785 PA0799 PA0804 PA0817 PA0829 PA0836 PA0838 PA0840 |
| | PA0842 PA0848 PA0853 PA0863 PA0879 PA0880 PA0882 PA0883 |
| | PA0936 PA0953 PA0954 PA0975 PA1014 PA1021 PA1022 PA1023 |
| | PA1027 PA1028 PA1032 PA1033 PA1045 PA1046 PA1047 PA1054 |
| | PA1056 PA1061 PA1062 PA1066 PA1127 PA1137 PA1163 PA1169 |
| | PA1171 PA1185 PA1187 PA1188 PA1202 PA1224 PA1225 PA1240 |
| | PA1252 PA1253 PA1254 PA1266 PA1284 PA1287 PA1292 PA1296 |
| | PA1303 PA1304 PA1306 PA1327 PA1330 PA1334 PA1344 PA1379 |
| | PA1389 PA1390 PA1391 PA1417 PA1470 PA1487 PA1499 PA1500 |
| | PA1535 PA1537 PA1538 PA1565 PA1576 PA1598 PA1601 PA1615 |
| | PA1617 PA1621 PA1622 PA1628 PA1631 PA1648 PA1649 PA1654 |
| | PA1655 PA1662 PA1678 PA1683 PA1736 PA1737 PA1739 PA1742 |
| | PA1748 PA1771 PA1813 PA1821 PA1827 PA1828 PA1829 PA1832 |
| | PA1833 PA1856 PA1860 PA1880 PA1881 PA1885 PA1890 PA1893 |
| | PA1914 PA1918 PA1919 PA1920 PA1962 PA1966 PA1984 PA1990 |
| | PA1991 PA1997 PA2022 PA2024 PA2035 PA2040 PA2062 PA2067 |
| | PA2069 PA2099 PA2105 PA2108 PA2119 PA2124 PA2125 PA2142 |
| | PA2152 PA2155 PA2158 PA2160 PA2162 PA2164 PA2188 PA2197 |
| | PA2199 PA2217 PA2231 PA2233 PA2237 PA2233 PA2263 PA2271 |
| | PA2275 PA2298 PA2302 PA2305 PA2310 PA2317 PA2324 PA2325 |
| | PA2326 PA2333 PA2346 PA2347 PA2348 PA2352 PA2355 PA2378 |
| | PA2379 PA2402 PA2411 PA2419 PA2475 PA2477 PA2478 PA2483 |
| | PA2491 PA2499 PA2535 PA2541 PA2546 PA2550 PA2552 PA2553 |
| | PA2554 PA2555 PA2578 PA2579 PA2580 PA2597 PA2603 PA2631 |
| | PA2634 PA2682 PA2698 PA2735 PA2745 PA2767 PA2815 PA2828 |
| | PA2841 PA2843 PA2865 PA2887 PA2888 PA2889 PA2890 PA2891 |
| | PA2892 PA2906 PA2918 PA2922 PA2934 PA2940 PA3035 PA3053 |
| | PA3106 PA3128 PA3132 PA3139 PA3141 PA3146 PA3147 PA3148 |
| | PA3149 PA3155 PA3156 PA3157 PA3158 PA3159 PA3172 PA3173 |
| | PA3217 PA3226 PA3232 PA3256 PA3277 PA3295 PA3301 PA3302 |
| | PA3312 PA3324 PA3328 PA3330 PA3368 PA3389 PA3426 PA3427 |
| | PA3429 PA3430 PA3437 PA3444 PA3454 PA3460 PA3498 PA3503 |
| | PA3504 PA3506 PA3507 PA3509 PA3511 PA3516 PA3529 PA3534 |
| | PA3535 PA3553 PA3554 PA3559 PA3567 PA3568 PA3579 PA3586 |
| | PA3593 PA3628 PA3659 PA3667 PA3682 PA3723 PA3759 PA3768 |
| | PA3774 PA3795 PA3798 PA3803 PA3817 PA3853 PA3859 PA3860 |
| | PA3883 PA3896 PA3913 PA3925 PA3957 PA3961 PA3968 PA3972 |
| | PA3994 PA4008 PA4019 PA4022 PA4026 PA4041 PA4073 PA4079 |
| | PA4088 PA4089 PA4097 PA4098 PA4114 PA4128 PA4131 PA4148 |
| | PA4162 PA4166 PA4167 PA4171 PA4175 PA4189 PA4190 PA4191 |
| | PA4198 PA4199 PA4202 PA4209 PA4217 PA4330 PA4342 PA4344 |
| | PA4361 PA4389 PA4401 PA4425 PA4431 PA4434 PA4435 PA4486 |
| | PA4512 PA4513 PA4576 PA4615 PA4619 PA4621 PA4676 PA4709 |
| | PA4715 PA4722 PA4724 PA4785 PA4786 PA4805 PA4819 PA4827 |
| | PA4832 PA4889 PA4899 PA4907 PA4943 PA4978 PA4979 PA4980 |
| | PA4986 PA4994 PA4995 PA5000 PA5004 PA5005 PA5007 PA5008 |
| | PA5020 PA5031 PA5048 PA5065 PA5084 PA5093 PA5127 PA5150 |
| | PA5175 PA5177 PA5181 PA5186 PA5187 PA5188 PA5190 PA5221 |
| | PA5234 PA5236 PA5238 PA5281 PA5309 PA5312 PA5313 PA5327 |
| | PA5384 PA5386 PA5390 PA5398 PA5432 PA5440 PA5445 PA5508 |
| | PA5514 PA5521 PA5522 PA5523 PA5524 PA5542 PA5546 PA5567 |
| Related to phage, | PA0257 PA0445 PA0616 PA0617 PA0618 PA0619 PA0620 PA0621 |
| transposon or | PA0622 PA0623 PA0624 PA0625 PA0626 PA0627 PA0628 PA0629 |
| plasmid | PA0630 PA0631 PA0632 PA0633 PA0634 PA0635 PA0636 PA0637 |
| | PA0638 PA0639 PA0640 PA0641 PA0642 PA0643 PA0644 PA0645 |
| | PA0646 PA0647 PA0648 PA0715 PA0717 PA0718 PA0719 PA0720 |
| | PA0721 PA0722 PA0723 PA0724 PA0725 PA0726 PA0727 PA0728 |
| | PA0909 PA0978 PA0979 PA0986 PA0987 PA1129 PA1937 PA1938 |
| | PA2319 PA2633 PA2690 PA3142 PA3143 PA3144 PA3434 PA3993 |
| | PA4797 |
| Secreted factors | PA0041 PA0044 PA0263 PA0763 PA0764 PA0765 PA0766 PA0843 |
| (toxins, enzymes, | PA0844 PA0852 PA0984 PA0985 PA1148 PA1150 PA1246 PA1247 |
| alginate) | PA1248 PA1249 PA1250 PA1431 PA1512 PA1707 PA1871 PA1876 |
| | PA1899 PA1900 PA1901 PA1902 PA1903 PA1904 PA1905 PA2191 |
| | PA2254 PA2255 PA2256 PA2257 PA2258 PA2259 PA2396 PA2399 |
| | PA2862 PA2863 PA2939 PA3319 PA3360 PA3478 PA3479 PA3540 |
| | PA3541 PA3542 PA3543 PA3544 PA3545 PA3546 PA3547 PA3548 |
| | PA3549 PA3550 PA3551 PA3724 PA3831 PA3841 PA3842 PA3866 |
| | PA4210 PA4211 PA4212 PA4213 PA4214 PA4215 PA4216 PA4225 |
| | PA4226 PA4228 PA4229 PA4230 PA4231 PA4446 PA4457 PA4528 |
| | PA5112 PA5261 PA5262 PA5267 PA5322 |
| Transcription, RNA | PA0428 PA0455 PA0584 PA0592 PA0733 PA0770 PA0903 PA0949 |
| processing and | PA0951 PA0963 PA1161 PA1294 PA2626 PA2738 PA2840 PA2859 |
| degradation | PA2861 PA2975 PA2976 PA3161 PA3246 PA3297 PA3308 PA3466 |
| | PA3528 PA3743 PA3744 PA3823 PA3831 PA3861 PA3950 PA4052 |
| | PA4238 PA4262 PA4264 PA4269 PA4270 PA4275 PA4544 PA4585 |
| | PA4720 PA4727 PA4740 PA4742 PA4745 PA4755 PA4853 PA4936 |
| | PA4937 PA4951 PA5197 PA5239 PA5334 PA5337 |
| Transcriptional | PA0032 PA0034 PA0037 PA0048 PA0056 PA0120 PA0123 PA0133 |
| regulators | PA0149 PA0150 PA0152 PA0155 PA0159 PA0163 PA0167 PA0175 |
| | PA0181 PA0191 PA0207 PA0217 PA0218 PA0225 PA0233 PA0236 |
| | PA0243 PA0248 PA0253 PA0268 PA0272 PA0275 PA0279 PA0289 |
| | PA0294 PA0306 PA0367 PA0376 PA0403 PA0416 PA0424 PA0436 |
| | PA0448 PA0456 PA0463 PA0471 PA0472 PA0475 PA0477 PA0479 |
| | PA0491 PA0513 PA0515 PA0527 PA0528 PA0533 PA0535 PA0547 |
| | PA0564 PA0576 PA0601 PA0610 PA0611 PA0652 PA0675 PA0676 |
| | PA0701 PA0707 PA0708 PA0739 PA0748 PA0756 PA0762 PA0763 |
| | PA0764 PA0765 PA0780 PA0784 PA0791 PA0797 PA0815 PA0816 |
| | PA0828 PA0831 PA0839 PA0864 PA0873 PA0876 PA0877 PA0893 |
| | PA0905 PA0906 PA0942 PA0961 PA1003 PA1015 PA1067 PA1097 |
| | PA1099 PA1109 PA1128 PA1136 PA1138 PA1141 PA1142 PA1145 |
| | PA1157 PA1159 PA1179 PA1182 PA1184 PA1196 PA1201 PA1223 |
| | PA1226 PA1229 PA1235 PA1241 PA1261 PA1264 PA1269 PA1283 |
| | PA1285 PA1290 PA1300 PA1301 PA1309 PA1312 PA1315 PA1328 |
| | PA1335 PA1347 PA1351 PA1359 PA1363 PA1364 PA1380 PA1397 |
| | PA1399 PA1403 PA1413 PA1422 PA1430 PA1431 PA1437 PA1455 |
| | PA1456 PA1484 PA1490 PA1504 PA1520 PA1526 PA1544 PA1570 |
| | PA1599 PA1603 PA1611 PA1619 PA1627 PA1630 PA1637 PA1653 |
| | PA1663 PA1704 PA1713 PA1738 PA1754 PA1759 PA1760 PA1776 |
| | PA1799 PA1826 PA1836 PA1850 PA1853 PA1859 PA1864 PA1884 |
| | PA1898 PA1911 PA1912 PA1945 PA1949 PA1961 PA1978 PA1980 |
| | PA1998 PA2005 PA2010 PA2016 PA2020 PA2028 PA2032 PA2047 |
| | PA2050 PA2051 PA2054 PA2056 PA2076 PA2082 PA2093 PA2094 |
| | PA2096 PA2100 PA2115 PA2118 PA2121 PA2123 PA2196 PA2206 |
| | PA2220 PA2227 PA2246 PA2258 PA2259 PA2267 PA2270 PA2273 |
| | PA2276 PA2277 PA2281 PA2299 PA2312 PA2316 PA2320 PA2332 |
| | PA2334 PA2337 PA2354 PA2359 PA2376 PA2383 PA2387 PA2388 |
| | PA2417 PA2426 PA2432 PA2447 PA2449 PA2467 PA2468 PA2469 |
| | PA2479 PA2488 PA2489 PA2492 PA2497 PA2510 PA2511 PA2519 |
| | PA2523 PA2534 PA2547 PA2551 PA2556 PA2572 PA2577 PA2583 |
| | PA2586 PA2588 PA2591 PA2601 PA2622 PA2657 PA2665 PA2681 |
| | PA2686 PA2692 PA2696 PA2704 PA2718 PA2758 PA2766 PA2798 |
| | PA2802 PA2809 PA2825 PA2834 PA2838 PA2846 PA2848 PA2849 |
| | PA2877 PA2879 PA2881 PA2885 PA2896 PA2897 PA2899 PA2917 |
| | PA2921 PA2930 PA2931 PA2957 PA3006 PA3027 PA3034 PA3045 |
| | PA3067 PA3077 PA3094 PA3122 PA3124 PA3133 PA3135 PA3174 |
| | PA3184 PA3192 PA3204 PA3215 PA3220 PA3225 PA3249 PA3260 |
| | PA3266 PA3269 PA3285 PA3321 PA3341 PA3346 PA3363 PA3364 |
| | PA3381 PA3398 PA3409 PA3410 PA3420 PA3423 PA3433 PA3458 |
| | PA3477 PA3508 PA3563 PA3565 PA3571 PA3574 PA3583 PA3587 |
| | PA3594 PA3599 PA3604 PA3622 PA3630 PA3678 PA3689 PA3699 |
| | PA3703 PA3711 PA3714 PA3721 PA3757 PA3771 PA3776 PA3778 |
| | PA3782 PA3830 PA3845 PA3895 PA3898 PA3899 PA3900 PA3921 |
| | PA3927 PA3932 PA3948 PA3965 PA3973 PA3995 PA4021 PA4032 |
| | PA4070 PA4074 PA4077 PA4080 PA4094 PA4101 PA4109 PA4120 |
| | PA4135 PA4145 PA4147 PA4157 PA4165 PA4174 PA4184 PA4185 |
| | PA4196 PA4203 PA4227 PA4288 PA4296 PA4315 PA4341 PA4381 |
| | PA4396 PA4436 PA4462 PA4493 PA4499 PA4508 PA4547 PA4581 |
| | PA4596 PA4600 PA4659 PA4723 PA4726 PA4764 PA4769 PA4776 |
| | PA4778 PA4781 PA4784 PA4787 PA4806 PA4831 PA4843 PA4853 |
| | PA4878 PA4885 PA4895 PA4896 PA4902 PA4906 PA4914 PA4983 |
| | PA4984 PA4987 PA4989 PA5029 PA5032 PA5059 PA5085 PA5105 |
| | PA5116 PA5125 PA5157 PA5166 PA5179 PA5189 PA5200 PA5218 |
| | PA5253 PA5255 PA5261 PA5274 PA5283 PA5293 PA5301 PA5308 |
| | PA5324 PA5342 PA5344 PA5356 PA5360 PA5364 PA5365 PA5374 |
| | PA5380 PA5382 PA5389 PA5403 PA5428 PA5431 PA5437 PA5438 |
| | PA5483 PA5499 PA5511 PA5525 PA5550 |
| Translation, post- | PA0008 PA0009 PA0018 PA0019 PA0067 PA0074 PA0090 PA0341 |
| translation | PA0355 PA0459 PA0538 PA0579 PA0580 PA0594 PA0668 PA0699 |
| modification, | PA0767 PA0768 PA0771 PA0837 PA0933 PA0956 PA1122 PA1165 |
| degradation | PA1481 PA1642 PA1670 PA1671 PA1710 PA1712 PA1793 PA1795 |
| | PA1796 PA1803 PA1805 PA1871 PA1928 PA1996 PA2071 PA2371 |
| | PA2476 PA2612 PA2617 PA2618 PA2619 PA2620 PA2725 PA2738 |
| | PA2739 PA2740 PA2741 PA2742 PA2743 PA2744 PA2748 PA2755 |
| | PA2851 PA2970 PA2973 PA2978 PA3007 PA3049 PA3083 PA3114 |
| | PA3134 PA3161 PA3162 PA3169 PA3227 PA3257 PA3262 PA3326 |
| | PA3482 PA3600 PA3601 PA3624 PA3635 PA3653 PA3655 PA3656 |
| | PA3657 PA3700 PA3701 PA3717 PA3724 PA3737 PA3742 PA3745 |
| | PA3802 PA3818 PA3823 PA3824 PA3831 PA3834 PA3871 PA3903 |
| | PA3984 PA3987 PA4138 PA4176 PA4237 PA4239 PA4240 PA4241 |
| | PA4242 PA4244 PA4245 PA4246 PA4247 PA4248 PA4249 PA4250 |
| | PA4251 PA4252 PA4253 PA4254 PA4255 PA4256 PA4257 PA4258 |
| | PA4259 PA4260 PA4261 PA4262 PA4263 PA4264 PA4265 PA4266 |
| | PA4267 PA4268 PA4271 PA4272 PA4273 PA4274 PA4277 PA4432 |
| | PA4433 PA4439 PA4446 PA4472 PA4482 PA4483 PA4484 PA4498 |
| | PA4542 PA4558 PA4559 PA4560 PA4563 PA4567 PA4568 PA4572 |
| | PA4665 PA4671 PA4672 PA4678 PA4741 PA4742 PA4743 PA4744 |
| | PA4768 PA4807 PA4808 PA4845 PA4850 PA4932 PA4934 PA4935 |
| | PA4941 PA4942 PA4945 PA5014 PA5018 PA5049 PA5051 PA5080 |
| | PA5134 PA5197 PA5224 PA5240 PA5254 PA5256 PA5315 PA5316 |
| | PA5440 PA5470 PA5474 PA5489 PA5569 PA5570 |
| Transport of small | PA0016 PA0029 PA0030 PA0073 PA0103 PA0119 PA0129 PA0136 |
| molecules | PA0137 PA0138 PA0151 PA0156 PA0157 PA0158 PA0162 PA0166 |
| | PA0184 PA0185 PA0186 PA0189 PA0192 PA0195 PA0196 PA0197 |
| | PA0198 PA0199 PA0203 PA0204 PA0205 PA0206 PA0215 PA0216 |
| | PA0220 PA0229 PA0235 PA0240 PA0241 PA0246 PA0273 PA0280 |
| | PA0281 PA0282 PA0283 PA0287 PA0295 PA0300 PA0301 PA0302 |
| | PA0303 PA0304 PA0313 PA0314 PA0322 PA0323 PA0324 PA0325 |
| | PA0326 PA0334 PA0337 PA0352 PA0374 PA0397 PA0425 PA0426 |
| | PA0427 PA0438 PA0443 PA0450 PA0458 PA0470 PA0476 PA0487 |
| | PA0602 PA0603 PA0604 PA0605 PA0606 PA0688 PA0693 PA0694 |
| | PA0703 PA0755 PA0783 PA0786 PA0789 PA0809 PA0811 PA0846 |
| | PA0860 PA0866 PA0884 PA0885 PA0886 PA0888 PA0889 PA0890 |
| | PA0892 PA0913 PA0917 PA0929 PA0930 PA0931 PA0950 PA0958 |
| | PA0969 PA0970 PA0971 PA0972 PA0973 PA1019 PA1025 PA1048 |
| | PA1051 PA1070 PA1071 PA1072 PA1074 PA1108 PA1113 PA1131 |
| | PA1144 PA1147 PA1183 PA1194 PA1207 PA1212 PA1236 PA1237 |
| | PA1238 PA1256 PA1257 PA1258 PA1260 PA1262 PA1271 PA1282 |
| | PA1286 PA1288 PA1297 PA1302 PA1313 PA1316 PA1339 PA1340 |
| | PA1341 PA1342 PA1361 PA1365 PA1386 PA1410 PA1418 PA1419 |
| | PA1425 PA1429 PA1435 PA1436 PA1475 PA1476 PA1477 PA1485 |
| | PA1491 PA1493 PA1496 PA1497 PA1507 PA1519 PA1541 PA1549 |
| | PA1569 PA1590 PA1626 PA1632 PA1633 PA1634 PA1635 PA1647 |
| | PA1650 PA1651 PA1682 PA1777 PA1783 PA1807 PA1808 PA1809 |
| | PA1810 PA1811 PA1819 PA1820 PA1848 PA1861 PA1862 PA1863 |
| | PA1882 PA1908 PA1910 PA1916 PA1922 PA1946 PA1947 PA1948 |
| | PA1958 PA1964 PA1971 PA1993 PA2006 PA2018 PA2019 PA2041 |
| | PA2042 PA2055 PA2058 PA2059 PA2060 PA2061 PA2068 PA2073 |
| | PA2079 PA2092 PA2113 PA2114 PA2135 PA2202 PA2203 PA2204 |
| | PA2210 PA2214 PA2252 PA2262 PA2278 PA2279 PA2291 PA2294 |
| | PA2295 PA2307 PA2308 PA2314 PA2322 PA2327 PA2329 PA2335 |
| | PA2338 PA2339 PA2340 PA2341 PA2350 PA2351 PA2390 PA2391 |
| | PA2397 PA2398 PA2408 PA2409 PA2435 PA2466 PA2472 PA2493 |
| | PA2494 PA2495 PA2500 PA2505 PA2520 PA2521 PA2522 PA2525 |
| | PA2526 PA2527 PA2528 PA2533 PA2558 PA2563 PA2592 PA2653 |
| | PA2678 PA2688 PA2700 PA2701 PA2711 PA2760 PA2811 PA2812 |
| | PA2835 PA2836 PA2837 PA2857 PA2911 PA2912 PA2913 PA2914 |
| | PA2923 PA2924 PA2925 PA2926 PA2933 PA2938 PA2983 PA2987 |
| | PA3000 PA3019 PA3038 PA3039 PA3136 PA3137 PA3153 PA3176 |
| | PA3186 PA3187 PA3188 PA3189 PA3190 PA3210 PA3211 PA3212 |
| | PA3228 PA3234 PA3236 PA3252 PA3253 PA3254 PA3264 PA3265 |
| | PA3268 PA3279 PA3280 PA3303 PA3313 PA3314 PA3315 PA3316 |
| | PA3336 PA3372 PA3373 PA3374 PA3375 PA3376 PA3377 PA3378 |
| | PA3379 PA3380 PA3382 PA3383 PA3384 PA3394 PA3407 PA3408 |
| | PA3441 PA3442 PA3443 PA3447 PA3448 PA3467 PA3512 PA3514 |
| | PA3521 PA3522 PA3523 PA3531 PA3538 PA3560 PA3561 PA3562 |
| | PA3573 PA3581 PA3588 PA3595 PA3597 PA3607 PA3608 PA3609 |
| | PA3610 PA3641 PA3660 PA3671 PA3672 PA3676 PA3677 PA3690 |
| | PA3709 PA3718 PA3739 PA3749 PA3753 PA3760 PA3761 PA3766 |
| | PA3781 PA3790 PA3837 PA3838 PA3839 PA3858 PA3865 PA3876 |
| | PA3877 PA3887 PA3888 PA3889 PA3890 PA3891 PA3901 PA3920 |
| | PA3926 PA3933 PA3936 PA3937 PA3938 PA3963 PA4023 PA4034 |
| | PA4037 PA4064 PA4072 PA4080 PA4096 PA4113 PA4126 PA4136 |
| | PA4137 PA4156 PA4158 PA4159 PA4160 PA4161 PA4168 PA4187 |
| | PA4192 PA4193 PA4194 PA4195 PA4206 PA4207 PA4208 PA4218 |
| | PA4221 PA4222 PA4223 PA4224 PA4225 PA4226 PA4228 PA4229 |
| | PA4230 PA4231 PA4233 PA4235 PA4289 PA4292 PA4334 PA4343 |
| | PA4355 PA4358 PA4365 PA4374 PA4375 PA4393 PA4455 PA4456 |
| | PA4461 PA4464 PA4466 PA4496 PA4497 PA4500 PA4501 PA4502 |
| | PA4503 PA4504 PA4505 PA4506 PA4514 PA4589 PA4590 PA4593 |
| | PA4594 PA4595 PA4597 PA4598 PA4599 PA4614 PA4616 PA4622 |
| | PA4628 PA4647 PA4654 PA4675 PA4687 PA4688 PA4706 PA4707 |
| | PA4710 PA4719 PA4770 PA4804 PA4821 PA4825 PA4859 PA4860 |
| | PA4861 PA4862 PA4887 PA4900 PA4903 PA4909 PA4910 PA4911 |
| | PA4912 PA4913 PA4981 PA4990 PA4997 PA5021 PA5030 PA5070 |
| | PA5074 PA5075 PA5076 PA5082 PA5094 PA5095 PA5096 PA5097 |
| | PA5099 PA5152 PA5153 PA5154 PA5155 PA5158 PA5159 PA5160 |
| | PA5165 PA5166 PA5167 PA5168 PA5169 PA5170 PA5207 PA5216 |
| | PA5217 PA5230 PA5231 PA5235 PA5252 PA5268 PA5282 PA5287 |
| | PA5291 PA5311 PA5317 PA5366 PA5367 PA5368 PA5370 PA5375 |
| | PA5376 PA5377 PA5434 PA5450 PA5451 PA5468 PA5476 PA5479 |
| | PA5500 PA5501 PA5503 PA5504 PA5505 PA5510 PA5518 PA5529 |
| | PA5530 PA5531 PA5548 |
| Two-component | PA0034 PA0178 PA0179 PA0408 PA0409 PA0413 PA0463 PA0464 |
| regulatory systems | PA0471 PA0600 PA0601 PA0756 PA0757 PA0928 PA0929 PA0930 |
| | PA1098 PA1099 PA1157 PA1158 PA1179 PA1180 PA1243 PA1335 |
| | PA1336 PA1396 PA1397 PA1437 PA1438 PA1456 PA1458 PA1611 |
| | PA1636 PA1637 PA1798 PA1799 PA1976 PA1979 PA1980 PA1992 |
| | PA2177 PA2479 PA2480 PA2523 PA2524 PA2571 PA2572 PA2583 |
| | PA2656 PA2657 PA2686 PA2687 PA2798 PA2809 PA2810 PA2824 |
| | PA2881 PA2882 PA3044 PA3045 PA3077 PA3078 PA3191 PA3192 |
| | PA3204 PA3206 PA3271 PA3346 PA3462 PA3604 PA3702 PA3704 |
| | PA3714 PA3878 PA3879 PA3946 PA3947 PA3948 PA3974 PA4032 |
| | PA4036 PA4101 PA4102 PA4112 PA4117 PA4196 PA4197 PA4293 |
| | PA4296 PA4380 PA4381 PA4396 PA4398 PA4493 PA4494 PA4546 |
| | PA4547 PA4725 PA4726 PA4776 PA4777 PA4781 PA4843 PA4856 |
| | PA4885 PA4886 PA4982 PA4983 PA5124 PA5125 PA5165 PA5166 |
| | PA5199 PA5200 PA5261 PA5262 PA5360 PA5361 PA5364 PA5483 |
| | PA5484 PA5511 PA5512 |

The sequence of the genes in Table 5 are also publicly available at http://www.pseudomonas.com/BrowseByFunctionU.asp

In certain embodiments the method includes the further step of determining whether the expression of a gene of one of the Function Classes set out below is down-regulated or absent:
Adaptation, protection;
Amino acid biosynthesis and metabolism;
Central intermediary metabolism;
DNA replication, recombination, modification and repair;
Nucleotide biosynthesis and metabolism;
Putative enzymes;
Related to phage, transposon or plasmid;
Transcription, RNA processing and degradation; and
Transport of small molecules.

In other embodiments, the method includes the further step of determining whether the expression of a gene of one of the Function Classes set out below is increased:
Adaptation, protection;
Amino acid biosynthesis and metabolism;
Antibiotic resistance and susceptibility;
Biosynthesis of cofactors, prosthetic groups and carriers;
Carbon compound catabolism;
Cell Wall and LPS;
Chaperones & heat shock proteins;
Chemotaxis;
DNA replication, recombination, modification and repair;
Energy metabolism;
Membrane proteins;
Motility & attachment;
Putative enzymes;
Related to phage, transposon or plasmid;
Secreted factors;
Transcriptional regulators;
Translation, post-translation modification, degradation;
Transport of small molecules; and
Two-component regulatory systems.

It will be understood that a gene of a particular “Function Class” means a gene of one of the Function Classes discussed in Table 5 for classification of P. aeruginosa genes. For example, a gene referred to by the phrase “a gene of Amino acid biosynthesis and metabolism Function Class”, means a gene in Table 1 under “Amino acid biosynthesis and metabolism”.

As discussed herein, the inventors have compared the expression of genes in infectious strains of P. aeruginosa having a common PFGE RFLP, described herein as “Pulsotype 1” or “P1” and “Pulsotype 2” or “P2” with the expression of genes of non infectious strains of P. aeruginosa. The inventors have found that the expression of genes of certain Function Classes in infectious strains is different to the expression of these genes in non infectious strains.

Thus in certain embodiments there is provided a method for determining whether an P. aeruginosa is infectious including assessing an opportunistic bacterium for the expression of:
one or more genes shown in Tables 1 to 4; or
one or more genes selected from the group consisting of: PA 0633, PA 0632, PA 0986, PA0637, PA0729, PA1617, PA0639, PA5471, PA5429, PA0636, PA0638, and PA3067; or
one or more genes selected from the group consisting of: PA0053, PA0632, PA0633, PA0634, PA0635, PA0636, PA0637, PA0638, PA0639, PA0730, PA1333, PA1393, PA3528, PA3531, PA3833, PA5429; or
the following genes: PA0632, PA0633, PA0634, PA0635, PA0636, PA0637, PA0638, PA0639; or
gene PA0729; or
the following genes: PA0364, PA1250, PA2587, PA3535, PA3904, PA4590, PA4738, PA4739, PA5220, PA5481, PA1001, PA1177, PA1431, PA1893, PA2007, PA2008, PA2009, PA2513, PA2588, PA3535, PA3904, PA3907, PA3923, PA4206, PA4498, PA4778, PA5061.

In other embodiments there is provided a method for determining whether an individual has been infected by an infectious strain of P. aeruginosa including:
obtaining a sample of P. aeruginosa bacterium from an individual;
assessing a P. aeruginosa bacterium of the sample for the expression of genes according to the above discussed method.

In certain embodiments the individual is a compromised individual. Specifically, the individual may have AIDS, cystic fibrosis or be a burns victim or a catheterized patient or patient subject to induced or artificial respiration.

In other embodiments there is provided a use of a nucleic acid capable of hybridising to:
one or more genes shown in Tables 1 to 4; or
one or more genes selected from the group consisting of: PA 0633, PA 0632, PA 0986, PA0637, PA0729, PA1617, PA0639, PA5471, PA5429, PA0636, PA0638, and PA3067; or
one or more genes selected from the group consisting of: PA0053, PA0632, PA0633, PA0634, PA0635, PA0636, PA0637, PA0638, PA0639, PA0730, PA1333, PA1393, PA3528, PA3531, PA3833, PA5429; or
the following genes: PA0632, PA0633, PA0634, PA0635, PA0636, PA0637, PA0638, PA0639; or
gene PA0729; or
the following genes: PA0364, PA1250, PA2587, PA3535, PA3904, PA4590, PA4738, PA4739, PA5220, PA5481, PA1001, PA1177, PA1431, PA1893, PA2007, PA2008, PA2009, PA2513, PA2588, PA3535, PA3904, PA3907, PA3923, PA4206, PA4498, PA4778, PA5061
   in the manufacture of means for determining whether an individual has been infected by an infectious strain of P. aeruginosa.

In other embodiments there is provided a use of a protein encoded by:
one or more genes shown in Tables 1 to 4; or
one or more genes selected from the group consisting of: PA 0633, PA 0632, PA 0986, PA0637, PA0729, PA1617, PA0639, PA5471, PA5429, PA0636, PA0638, and PA3067; or
one or more genes selected from the group consisting of: PA0053, PA0632, PA0633, PA0634, PA0635, PA0636, PA0637, PA0638, PA0639, PA0730, PA1333, PA1393, PA3528, PA3531, PA3833, PA5429; or
the following genes: PA0632, PA0633, PA0634, PA0635, PA0636, PA0637, PA0638, PA0639; or
gene PA0729; or
the following genes: PA0364, PA1250, PA2587, PA3535, PA3904, PA4590, PA4738, PA4739, PA5220, PA5481, PA1001, PA1177, PA1431, PA1893, PA2007, PA2008, PA2009, PA2513, PA2588, PA3535, PA3904, PA3907, PA3923, PA4206, PA4498, PA4778, PA5061
   in the manufacture of means for determining whether an individual has been infected by an infectious strain of P. aeruginosa.

In other embodiments there is provided a use of an antibody capable of binding to a protein encoded by:
one or more genes shown in Tables 1 to 4; or
one or more genes selected from the group consisting of: PA 0633, PA 0632, PA 0986, PA0637, PA0729, PA1617, PA0639, PA5471, PA5429, PA0636, PA0638, and PA3067; or
one or more genes selected from the group consisting of: PA0053, PA0632, PA0633, PA0634, PA0635, PA0636, PA0637, PA0638, PA0639, PA0730, PA1333, PA1393, PA3528, PA3531, PA3833, PA5429; or
the following genes: PA0632, PA0633, PA0634, PA0635, PA0636, PA0637, PA0638, PA0639; or
gene PA0729; or
the following genes: PA0364, PA1250, PA2587, PA3535, PA3904, PA4590, PA4738, PA4739, PA5220, PA5481, PA1001, PA1177, PA1431, PA1893, PA2007, PA2008, PA2009, PA2513, PA2588, PA3535, PA3904, PA3907, PA3923, PA4206, PA4498, PA4778, PA5061
in the manufacture of means for determining whether an individual has been infected by an infectious strain of P. aeruginosa.

In another embodiment there is provided a process for determining whether an opportunistic bacterium is infectious including assessing whether the bacterium is adapted to produce a bio-film. Typically the assessment is undertaken by assessing the expression of one or more of the following genes:
0158, 0524, 0729, 2138, 2396, 2412, 2427, 2587, 2662, 3371, 5217, 5481, 0099, 0447, 0672, 0676, 1123, 1173, 1177, 1217, 1218, 1219, 1259, 1318, 1349, 1535, 1555, 1556, 1557, 1558, 1790, 1892, 1893, 1910, 2034, 2095, 2122, 2383, 2384, 2404, 2413, 2508, 2619, 2687, 2831, 2907, 3126, 3535, 4009, 4182, 4206, 4588, 4658, 4810, 4909, 4913, 4973, 5188, 5380, 5460, 5471 and 5510.

In certain embodiments there is provided a kit for determining whether an opportunistic bacterium is infectious including:
a reagent for assessing an opportunistic bacterium for expression of a gene selected from the group consisting of PA0632, PA0633, PA0636, PA0637, PA0638, PA0639, PA0986, PA1617, PA 3067, PA5429, and PA5471; and
written instructions for using the reagent to assess an opportunistic bacterium for expression of the gene.

The reagent may be a nucleic acid having a sequence that is complimentary to the sequence of one of the above genes. It may also be an antibody or other protein for detecting the expression of a product of one of the above genes.

### EXAMPLES

### Materials and Methods

### Bacterial Strain Selection

P. aeruginosa was previously isolated from the sputum of cystic fibrosis patients, identified through established biochemical and morphological methods and stored at −80° C. in screw-cap vials containing “Protect” cryopreservation beads (Technical Service Consultants Ltd, UK). They were subsequently classified as belonging to a pulsotype or unique group on the basis of their banding pattern after PFGE of SpeI-generated genomic digests. For this study, a selection of PI and unique isolates were chosen as pairs, based on similar antibiotic resistance profiles and the patients' age and forced expiratory volume in 1 second (FEV-1). Four pairs of PI and unique isolates were thus selected (Table 6). This matching, while not perfect, reduced the impact of other health-related variables and enabled analysis of subgroups based on patient age.

### RNA Extraction and Purification

Isolates were resuscitated by removing a bead from “Protect” and streaking for isolated colonies onto fresh HBA plates for incubation at 35° C. Isolated colonies were usually visible after 18-36 hrs of incubation, depending on strain, and several were then picked into 2 ml L-broth and grown to mid-log phase (16-18 hrs; optical density (OD₆₀₀)=0.5±0.05) with shaking (200 rpm) at 35° C.

Total RNA from each strain was stabilized for isolation using RNAprotect Bacteria Reagent and extracted using the RNeasy Mini Purification kit (QIAGEN Pty Ltd, Victoria, Australia) as per the manufacturer's protocols. Briefly, 500 μl aliquots of mid-log phase cells were stabilized in 1000 μl of RNAprotect and lysed using 1 mg/ml lysozyme (L6876, Sigma-Aldrich, Mo. USA). The extracts were treated for DNA contamination using the RNase-Free Dnase Set (QIAGEN) and the RNA eluted in 30 μl RNase-free water. RNA concentration was determined by absorbance at 260 nm, with a minimum concentration of ca. 500 ng/μl required to proceed to cDNA synthesis. The quality of the extracted RNA and presence of any residual DNA were checked by formaldehyde agarose gel electrophoresis.

cDNA Synthesis, Fragmentation and Labeling

cDNA was synthesized, fragmented and labeled as per the Affymetrix GeneChip Expression Analysis Technical Manual (Affymetrix Corp., CA. USA) (http://www.Affymetrix.comsupport/technical/byproduct.affx?product=paeruginosa). Briefly, cDNA was synthesized by using random hexamers as primers for reverse transcription (Invitrogen Australia Pty Ltd, Victoria, Australia). The primers were annealed (70° C. for 10 min, followed by 25° C. for 10 min) to 10 μg total RNA. Polyadenylated control transcripts (130 pM) were added to each sample (Affymetrix) to monitor transcriptional efficiency and array performance. Transcripts were extended with SuperScript II reverse transcriptase (Invitrogen) (25° C. for 10 min, 37° C. for 60 min 42° C. for 60 min and 70° C. for 10 min). Residual RNA was removed by alkaline treatment followed by neutralization and the cDNA was purified with a MinElute™ PCR Purification kit (QIAGEN). Purified cDNA was fragmented using DNase I (Amersham Biosciences, NSW, Australia) and the fragments 3′-end-labeled using GeneChip® DNA Labeling Reagent (Affymetrix).

### Microarray Processing

Fragmented labelled DNA was sent to an external facility, (Australian Genome Research Facility [AGRF], Melbourne, Australia) for microarray analysis, where fragmentation quality was checked using a Bioanalyser 2100 (Agilent GmbH, Waldbronn, Germany) and the NanoChip protocol (Agilent). Samples that contained fragmented DNA of ca. 100 bp length were prepared for hybridization to the Pseudomonas aeruginosa Genome Array (Affymetrix) by adding 3-7 μg DNA to a probe cocktail that included 1×Hybridization Buffer (100 mM MES, 1 mM NaCl, 20 mM EDTA, 0.01% Tween-20), 0.1 mg/ml Herring Sperm DNA, 0.5 mg/ml BSA, and 7% DMSO (Sigma-Aldrich).

An initial ‘test3’ array (Affymetrix) comprising 100 housekeeping genes, was carried out on the first batch of 5 samples, to determine DNA suitability for the full array. In this case, a 220 μl volume of probe cocktail was prepared for each sample; with 90 μl being used in the ‘test3’ array and the remainder in the full array. For subsequent full arrays, a total volume of 130 μl was prepared for each sample and loaded onto a P. aeruginosa chip via a self sealing aperture. The chip was hybridized at 50° C. for 16 h in a hybridization oven with a rotating wheel at 60 rpm. After hybridization, the chip was washed using the Pseudomonas fluidics script in the Affymetrix Fluidics Station 450. Upon completion of the washing, the chips were scanned using the Affymetrix GeneChip Scanner 3000 with a 532 nm solid-state laser to excite probe array fluorophores, producing an emission wavelength of 570 nm. The scanner operating software, GCOS, then converted the signal on the chip into a DAT file, which was used in generating subsequent CEL and CHP files for analysis. Most P. aeruginosa genes were expressed above the detection level of the Affymetrix microarray technology (average of present calls=84%).

### Data Analysis

Microarray data were initially analyzed with the ArrayAssist package (Stratagene Corp. CA. USA). Affymetrix .CEL files were pre-processed and normalized with the PLIER algorithm (Affymetrix). Since the number of arrays being analyzed was small no correction to the p-values was made. While the resultant list of differentially expressed genes will thus contain a number of false positives, we were willing to accept this at this stage in the analysis in order to avoid false negatives. A more robust analysis using statistical methods was carried out later as described below. In this first analysis, genes were determined to be differentially expressed if they had an uncorrected p-value less than 0.1 and a fold change in expression of greater than 1.4.

A robust statistical analysis of the microarray data was done using packages from the Bioconductor software suite. Affymetrix .CEL files were processed using the affy package to normalize the data, thus adjusting for variation introduced during sample preparation, array manufacture, and array processing. The robust multi-array average (RMA) method was used. This incorporates probe level background-correction, quantile normalization, and the use of a linear model to extract a final expression measure for each gene on each array.

The resulting expression measures were then used to determine differential expression using an empirical Bayes approach within the limma package. The false discovery rate method of Benjamini and Hochberg was controlled to reduce false positives introduced by multiple simultaneous inference. The empirical Bayes approach ranks the genes in order of decreasing likelihood of differential expression using a B-statistic (log-odds of differential expression). A specific cut-off for determining the differentially expressed genes is generally not appropriate with this type of analysis; however, for this study we used a positive B-statistic, i.e., a greater than 50-50 chance that the gene in question is differentially expressed, as a guide for statistically significant differential expression.

In addition to the statistical analysis above, we also considered those genes with a simple two-fold change in gene expression. These genes were drawn from the results of the limma analysis where the log-differential expression ratio was greater than 1 (2-fold upregulated) or less than −1 (2-fold down-regulated).

### Results

### Bacteriophage and Biofilm-Related Genes are Differentially Expressed in PI

With the unique isolates as a reference, we found that 87 genes and three intergenic regions of the 5900 genes on the P. aeruginosa chip (1.5% of the total number on the array) were differentially expressed (p<0.1) in all PI isolates (Table 7). Of the total, 83 were homologs of PA01 genes, four were homologous to genes located on glycosylation islands from P. aeruginosa strain PAK and three corresponded to intergenic regions on PA01. 71 of the 87 genes identified were upregulated and 16 were downregulated. The most prominent gene function class was related to phage, transposons or plasmids. 16.9% of the genes in the P. aeruginosa PI genome that were differentially expressed came from this class, the largest proportion of any of the ‘Gene Function’ classes determined by the Pseudomonas aeruginosa Community Annotation Project (PseudoCAP at: www.pseudomonas.com). Amongst these was PA0729, which showed the greatest upregulation of all genes, being ca. 20-fold higher in PI compared to the unique isolates. This gene belongs to a cluster of genes related to the filamentous bacteriophage Pf1, eleven of which are found in P. aeruginosa PA01. A contiguous pair of putative phage related genes, PA986 and PA987, were also significantly upregulated (ca.7-fold), while another cluster of putative phage/transposon or plasmid genes, PA0632-PA0639, were significantly downregulated (−3.4 to −11.2 fold) in the PI isolates. PA0632 has no known function, but PA0633 is also known as VF2 protein and has 54% homology to phage φCTX, while PA0636, PA0637 and PA0639 have 50%, 53% and 56% homology to tail proteins of bacteriophage N15. This cluster had been reported as being absent in the Liverpool epidemic strains LES400 and 431. In order to ascertain if this was also the case with PI, we attempted to amplify this region in all eight PI and unique isolates, using PCR primers to 2 regions of the cluster; PA0632-0633 and PA0636-0637. Both of these regions could be amplified from all unique isolates but not from any of the PI isolates, suggesting that these phage-related genes have been lost in PI (data not shown).

A statistical comparison of PI against unique isolates using BIOCONDUCTOR, to further verify our initial analysis, also showed a large proportion of differentially regulated bacteriophage genes. Four arrays derived from the PI isolates were compared with the arrays of four unique isolates, and genes with a positive B-statistic are shown in Table 8. The results show that while there are fewer genes overall than seen with the unpaired t-test (Table 7), all but one of the genes with a positive B statistic in Table 8 is also present in Table 7. Two genes identified above as highly upregulated, PA0986 and PA0729, are also upregulated here by ca. 8 and 13-fold respectively. Eight genes are downregulated, notably including the abovementioned region of the genome from PA0632 through to PA0639; as well as aspartate ammonia lyase (aspA). All of the downregulated genes showed a fold change greater than 2-fold.

Differential expression of quorum sensing (QS) and biofilm-regulated genes is also a feature of PI. Three genes associated with QS were differentially expressed in PI: PA0413, PA1250 (aprI) and PA5451 (wzm). With respect to genes differentially expressed during biofilm formation, Table 9A shows that 16 of the 83 PA01-matched genes identified as differentially regulated in all PI were also differentially expressed during PA01 biofilm formation. In the case of PI from older patients, 51 of the 576 differentially expressed PA01 genes were also differentially expressed in biofilms. A number of these were also differentially expressed.

An important comparison has been made possible with recent publication of genomic expression in the transmissible strains LES400 and LES431. In that study, 32 and 133 genes were upregulated in LES400 and LES431, respectively, compared to PA01, when grown in planktonic culture. Table 10 shows that a number of these are biofilm or QS-regulated genes that are also upregulated in all PI. They include aprI, pqsH (PA2587-regulated by the LasI/R quorum-sensing system) and pra (PA4590-a surface active emulsifying agent similar in function to the RhIR rhamnolipid). When differentially expressed PI genes from the older patients were compared with the two LES strains, 20 genes were found to be differentially regulated, including rsaL QS repressor (upregulated in both PI and LES), mexH (upregulated in both) and the QS-regulated phnA, a Pseudomonas quinolone system (PQS) signal precursor (downregulated in PI, upregulated in LES).

It should also be noted however, that a significant number of the genes differentially regulated in all PI are not among those differentially regulated in the LES strains and vice versa. Those differentially regulated in PI but not LES400 or 431 include biofilm and QS-regulated genes such as norB (PA0524), rnt (PA3528), bfrB (PA3531), aspA (PA5429), wbpY (PA5448), and wzm (PA5451). Genes differentially regulated in LES400 or 431 but not PI, are more numerous, and include several nitrate reductase (nap), phenazine biosynthesis (phz), hydrogen cyanide (hcn) genes as well as components of the las and rhl systems. Therefore there are numerous differences in number and function of differentially regulated genes between the transmissible PI and LES isolates.

### PI in Older Chronically Infected Patients Exhibits Greater Differential Expression

The number of differentially expressed genes in PI compared to unique isolates from the older patients (average age 31.5 yrs; average length of known P. aeruginosa infection: 11 yrs), was substantially greater than that for PI versus unique isolates from younger patients (average age 21.8 yrs; average length of known P. aeruginosa infection: 5 yrs). While they were known to have P. aeruginosa infection, the pulsotype status of the patients could not however be confirmed for more than the last 12 months of sputum collection. 584 genes (10% of the genes on the array) were differentially regulated in the PI isolates from older patients as opposed to just 16 genes (0.3% of the genes on the array) in the PI from the younger patients. Of the genes from older patients, 575 were homologous to PA01 genes and nine to strain PAK genes. 521 genes were upregulated and 63 downregulated (Tables 9A and 9B Supplementary data). Several genes, including the phage-related genes PA0729, PA0632, PA0636 and PA0637, are present amongst differentially expressed PI genes from both younger and older patients, testifying to their overall importance in PI. The differentially regulated PI genes from older patients also underwent greater fold upregulation than PI genes from younger patients; for example upregulation of PA0729 is four times higher in the older PI group compared to the younger PI group (44fold vs. 11.5fold) and three times higher in both aprI (11.1 vs. 3.7fold) and PA1291 (8fold vs. 2.1fold).

Classification of these differentially regulated genes by functional group as per PseudoCAP showed a bias towards certain groups. 20.3% of carbon compound catabolism genes were differentially regulated (18% up, 2.3% down) in the PI isolates from older patients compared to none for those from the younger patients. Other highly differentially expressed groups in older patients included: fatty acid and phospholipid metabolism (15.6%), antibiotic resistance (13%), transcriptional regulators (12.8%), transport of small molecules, including 19 ABC transporters (11.9%) and secreted factors (10.8%). In contrast, genes from certain other functional groups showed little or no differential regulation; examples include cell wall/LPS/capsule genes (0.7%), motility and attachment genes (1.9%), genes involved in transcription, RNA processing and degradation (1.9%), and protein secretion/export apparatus genes (2.0%). Phage/transposon/plasmid related genes stand out as being equally differentially expressed in PI from both younger and older patients (9.2% and 10.8% respectively), hinting at this groups' overall importance to the PI strain. The upregulated genes belonging to the transport of small molecules group include 19 components of ABC transporters, which are essential in bacterial adaptation to new environments as well as increased virulence associated with nutrient and metal ion uptake.

### Differential Expression of Virulence Genes in PI

Amongst other differentially expressed genes were nine genes involved in the transport of small molecules, including several ABC and efflux transporters and bacterioferritin (BfrB); and seven membrane proteins. This suggests that ionic exchange within the cell and with the surroundings is more active in PI. Also noteworthy is the upregulation of four glycosylation island genes (Pae orfD, H, I, and M) from P. aeruginosa strain PAK. These genes were present in all PI, but gave absent fluorescence calls in all unique isolates except MV-1a, signifying the possible acquisition of PAK gene island genes by PI.

TABLE 6
List of P. aeruginosa CF isolates used in this study, together with their pulsotype classification
and antibiotic resistance. The isolates are displayed as they were paired for matching for: antibiotic
resistance, age and forced expiratory volume (FEV-1) reading of the patient from whom the isolate was obtained.

| | | Antibiotic Resistance | | | | | | | Patient | Patient |
|---|---|---|---|---|---|---|---|---|---|---|
| Isolate | Type | Ciprofloxacin | Cefepime | Timentin | Meropenem | Gentamycin | Amikacin | Colistin | Age* | FEV-1† |
| 1 | PI | R | R | S | S | R | R | S | 29 | 1.15/40% |
| 2 | unique | R | R | S | R | R | R | S | 29 | 1.68/58% |
| 3 | PI | R | R | S | S | R | R | S | 31 | 0.88/29% |
| 4 | unique | R | R | R | R | R | R | S | 37 | 1.60/55% |
| 5 | PI | R | R | S | S | R | R | S | 24 | 2.18/58% |
| 6 | unique | R | R | R | S | R | R | S | 22 | 2.75/59% |
| 7 | PI | R | R | R | R | R | R | S | 22 | 1.28/28% |
| 8 | unique | S | R | S | S | R | R | S | 19 | 2.70/58% |

*Age at sputum collection.
†Forced expiratory volume in 1 second/percentage of predicted volume.

TABLE 7
Genes differentially expressed between transmissible (PI) and non-transmissible (unique)
isolates according to description and function (p < 0.1).

| Gene | Fold | Description | Function |
|---|---|---|---|
| PA0053 | −6.5 | Hypothetical protein | |
| PA0100 | 4.5 | Hypothetical protein | |
| PA0158 | 1.9 | Probable RND efflux transporter | Membrane proteins; Transport of small |
| | | | molecules |
| PA0364 | 2.6 | Probable oxidoreductase | Putative enzymes |
| PA0365 | 2.3 | Hypothetical protein | Membrane proteins |
| PA0413 pilL | 2.2 | Still-frameshift component of chemotactic signal | Motility & Attachment; Chemotaxis; |
| | | transduction chpA | Two-component regulatory systems |
| PA0498 | 3.2 | Hypothetical protein | |
| PA0524 | 2.0 | Nitric oxide reductase subunit B | Energy metabolism |
| norB | | | |
| PA0632 | −4.1 | Hypothetical protein-phage, transposon or plasmid | |
| | | related | |
| PA0633 | −11.2 | Hypothetical protein-phage, transposon or plasmid | |
| | | related | |
| PA0634 | −4.8 | Hypothetical protein-phage, transposon or plasmid | |
| | | related | |
| PA0635 | −3.7 | Hypothetical protein-phage, transposon or plasmid | |
| | | related | |
| PA0636 | −6.7 | Hypothetical protein-phage, transposon or plasmid | |
| | | related | |
| PA0637 | −4.4 | Hypothetical protein-phage, transposon or plasmid | |
| | | related | |
| PA0638 | −5.0 | Hypothetical protein-phage, transposon or plasmid | |
| | | related | |
| PA0639 | −3.4 | Hypothetical protein-phage, transposon or plasmid | |
| | | related | |
| PA0729 | 20.6 | Hypothetical protein-related to bacteriophage Pf1 | |
| PA0730 | −3.6 | Probable transferase | Putative enzymes |
| PA0866 | 2.3 | Aromatic amino acid transport protein AroP2 | Transport of small molecules |
| aroP2 | | | |
| PA0986 | 6.8 | Hypothetical protein-phage, transposon or plasmid | |
| | | related | |
| PA0987 | 7.3 | Hypothetical protein-phage, transposon or plasmid | |
| | | related | |
| PA1129 | 2.6 | Probable fosfomycin resistance protein | Antibiotic resistance & susceptibility |
| PA1214 | 1.6 | Hypothetical protein | |
| PA1250 aprI | 3.7 | Alkaline proteinase inhibitor AprI | Secreted Factors (toxins, enzymes, |
| | | | alginate) |
| PA1291 | 3.3 | Hypothetical protein | |
| PA1333 | −3.4 | Hypothetical protein | |
| PA1393 | −2.9 | Adenosine 5-phosphosulfate kinase | Central intermediary metabolism; |
| cysC | | | Nucleotide biosynthesis and |
| | | | metabolism; Amino acid biosynthesis |
| | | | and metabolism. |
| PA1395 | 2.4 | Hypothetical protein | |
| PA1617 | 4.4 | Probable AMP-binding enzyme | Putative enzymes |
| PA1629 | 2.0 | Probable enoyl-CoA hydratase/isomerase | Carbon compound catabolism |
| PA1759 | 1.9 | Probable transcriptional regulator | Transcriptional regulators |
| PA1929 | 3.4 | Hypothetical protein | |
| PA2037 | 2.9 | Hypothetical protein | |
| PA2104 | 3.9 | Probable cysteine synthase | Amino acid biosynthesis/metabolism |
| PA2105 | 4.4 | Probable acetyltransferase | Putative enzymes |
| PA2106 | 3.7 | Hypothetical protein | |
| PA2138 | 2.3 | Probable ATP-dependent DNA ligase | DNA replication, recombination, |
| | | | modification and repair |
| PA2384 | 2.1 | Hypothetical protein | |
| PA2387 | 4.4 | Probable sigma70 factor-ECF family. | Transcriptional regulators |
| PA2396 | 3.2 | Pyoverdine synthetase F | Adaptation, protection; Secreted |
| pvdF | | | Factors (toxins, enzymes, alginate) |
| PA2409 | 1.8 | Probable permease of ABC transporter | Membrane proteins; Transport of small |
| | | | molecules |
| PA2412 | 2.3 | Hypothetical protein | |
| PA2423 | 2.8 | Hypothetical protein | |
| PA2427 | 3.3 | Hypothetical protein | |
| PA2435 | 1.8 | Probable cation transporting P-type ATPase | Membrane proteins; Transport of small |
| | | | molecules |
| PA2587 | 2.7 | Probable FAD-dependent monooxygenase | Biosynthesis of cofactors, prosthetic |
| pqsH | | | groups and carriers |
| PA2662 | 1.7 | Hypothetical protein | |
| PA2663 | 1.8 | Hypothetical protein | |
| PA3132 | 1.9 | Probable hydrolase | Putative enzymes |
| PA3318 | 3.3 | Hypothetical protein | |
| PA3320 | 2.2 | Hypothetical protein | Membrane proteins |
| PA3354 | 1.8 | Hypothetical protein | |
| PA3358 | 2.1 | Hypothetical protein | Membrane proteins |
| PA3371 | 2.2 | Hypothetical protein | |
| PA3429 | 1.9 | Probable epoxide hydrolase | Putative enzymes |
| PA3528 rnt | −2.5 | RibonucleaseT-DNA-replication, transcription | DNA replication, recombination, |
| | | | modification and repair. Transcription, |
| | | | RNA processing and degradation |
| PA3531 bfrB | −3.9 | Bacterioferritin | Transport of small molecules, |
| | | | adaptation, protection |
| PA3535 | 1.8 | Probable serine protease | Putative enzymes |
| PA3833 | −3.0 | Hypothetical protein | |
| PA3860 | 2.0 | Probable AMP-binding enzyme | Putative enzymes |
| PA3904 | 3.1 | Hypothetical protein | |
| PA3905 | 2.5 | Hypothetical protein | |
| PA4075 | 2.2 | Hypothetical protein | |
| PA4190 | 2.4 | Probable FAD-dependent monooxygenase | Putative enzymes |
| pqsL | | | |
| PA4349 | 1.8 | Hypothetical protein | |
| PA4386 | 1.8 | Chaperone and heat shock protein | Chaperones & heat shock proteins |
| groES | | | |
| PA4590 pra | 2.5 | Protein activator Pra | Carbon compound catabolism; |
| | | | Transport of small molecules |
| PA4677 | 3.0 | Hypothetical protein | |
| PA4738 | 3.0 | Hypothetical protein | |
| PA4739 | 3.5 | Hypothetical protein | |
| PA4908 | 2.8 | Hypothetical protein | |
| PA4909 | 2.8 | Probable ATP-binding component of ABC transporter | Transport of small molecules |
| PA4994 | 2.2 | Probable acyl-CoA dehydrogenase | Putative enzymes |
| PA5217 | 2.4 | Probable binding component-ABC transporter | Transport of small molecules |
| PA5220 | 4.2 | Hypothetical protein | |
| PA5352 | 2.5 | Hypothetical protein | |
| PA5429 | −2.1 | Aspartate ammonia lyase AspA | Amino acid biosynthesis and |
| aspA | | | metabolism |
| PA5448 | 1.9 | Glycosyltransferase WbpY | Cell wall/LPS/capsule |
| wbpY | | | |
| PA5451 | 1.9 | Membrane subunit of A-band LPS efflux transporter | Membrane proteins; Transport of small |
| wzm | | | molecules; Cell wall/LPS/capsule |
| PA5470 | 3.9 | Probable peptide chain release factor | Translation, post-translational |
| | | | modification, degradation |
| PA5471 | 4.5 | Hypothetical protein | |
| PA5481 | 3.1 | Hypothetical protein | |
| PA5542 | 2.6 | Hypothetical protein | Putative enzymes |
| Pae_orfD | 3.7 | Putative 3-oxoacyl reductase-acyl carrier protein-PAK | |
| Pae_orfH | 3.9 | Unknown ORF from glycosylation island-strain PAK | |
| Pae_orfI | 3.5 | Unknown ORF from glycosylation island-strain PAK | |
| Pae_orfM | 4.1 | Putative aldolase from glycosylation island-strain PAK | |

Genes are listed in order of locus number.
Gene ID in bold = Contiguous genes-putative operon.

TABLE 8
Genes showing statistically significant differential expression
between transmissible (PI) and unique) isolates. Statistical analysis
was undertaken using the Bioconductor Affy and limma packages.
The p-value has been corrected using the false discovery rate. Genes
are listed in order of decreasing B-statistic.

| | | | p- | |
|---|---|---|---|---|
| Probe | Description | Fold | value | B-statistic |
| PA0633 | Phage-related hypothetical | −8.6 | 0.014 | 3.283 |
| PA0632 | Phage-related hypothetical | −4.2 | 0.031 | 2.494 |
| PA0986 | Conserved phage-related hypothetical | 8.1 | 0.031 | 2.294 |
| PA0637 | Conserved phage-related hypothetical | −3.9 | 0.033 | 2.078 |
| PA0729 | Hypothetical | 12.7 | 0.038 | 1.872 |
| PA1617 | Probable AMP-binding enzyme | 4.3 | 0.042 | 1.692 |
| PA0639 | Conserved phage-related hypothetical | −3.9 | 0.106 | 0.972 |
| PA5471 | Hypothetical protein | 3.2 | 0.114 | 0.830 |
| PA5429 | Aspartate ammonia-lyase (aspA) | −2.2 | 0.122 | 0.703 |
| aspA | | | | |
| PA0636 | Phage-related hypothetical | −6.6 | 0.140 | 0.531 |
| PA0638 | Probable bacteriophage protein | −3.6 | 0.174 | 0.250 |
| PA3067 | Probable transcriptional regulator | −2.7 | 0.174 | 0.236 |

TABLE 9A
Genes differentially expressed both during PA01 biofilm formation and
in all PI isolates during planktonic growth. Some have been identified
as biofilm associated in other P. aeruginosa biofilm studies using PA01.

| | Regulation* | | | | |
|---|---|---|---|---|---|
| ORF | 1. | 2. | 3. | 4. | Description |
| PA0158 | + | − | + | + | Probable RND efflux transporter |
| PA0524 norB | + | + | ND | + | Nitric oxide reductase subunit B |
| PA0729 | + | +† | ND | ND | Related to bacteriophage Pf1 |
| PA2138 | + | − | ND | ND | Probable ATP-dependent |
| | | | | | DNA ligase |
| PA2384 | + | + | ND | ND | Hypothetical protein |
| PA2396 pvdF | + | + | ND | ND | Pyoverdine synthetase F |
| PA2412 | + | + | ND | ND | Hypothetical protein |
| PA2427 | + | + | ND | ND | Hypothetical protein |
| PA2587 | + | − | − | − | Probable FAD-dependent |
| pqsH | | | | | monooxygenase |
| PA2662 | + | + | ND | ND | Hypothetical protein |
| PA3371 | + | − | + | − | Hypothetical protein |
| PA3535 | + | − | + | ND | Probable serine protease |
| PA4909 | + | + | ND | ND | Probable ATP-binding comp.- |
| | | | | | ABC transporter |
| PA5217 | + | − | ND | ND | Probable binding comp.- |
| | | | | | ABC transporter |
| PA5471 | + | + | ND | ND | Hypothetical protein |
| PA5481 | + | − | + | ND | Hypothetical protein |

*Upregulation (+), Downregulation (−), ND-No differential expression. 1. PI-this study 2. Separate study 3. Separate study 4. Separate study.
†Negative at 24 h, positive thereafter.

TABLE 9B
Genes differentially expressed during biofilm formation and in PI isolates
from older patients during planktonic growth. Some have been identified
as biofilm associated in other P. aeruginosa biofilm studies using PA01.

| | Regulation* | | | | |
|---|---|---|---|---|---|
| ORF | 1. | 2. | 3. | 4. | Description |
| PA0099 | + | + | ND | ND | Hypothetical protein |
| PA0447gcdH | − | − | − | + | Glutaryl-CoA dehydrogenase |
| PA0672hemO | + | + | ND | ND | Heme oxygenase |
| PA0676 | + | + | ND | ND | Probable transmembrane sensor |
| PA0729 | + | +† | ND | ND | Related to bacteriophage Pf1 |
| PA1123 | − | + | ND | ND | Hypothetical protein |
| PA1173 napB | − | + | + | ND | Cytochrome c-type protein precursor |
| PA1177 napE | − | + | + | − | Periplasmic nitrate reductase protein |
| PA1217 | + | + | + | + | Probable 2-isopropylmalate synthase |
| PA1218 | + | + | + | − | Hypothetical protein |
| PA1219 | + | + | − | + | Hypothetical protein |
| PA1259 | + | + | ND | ND | Hypothetical protein |
| PA1318 cyoB | + | − | + | + | Cytochrome o ubiquinol oxidase subunit I |
| PA1349 | + | + | ND | ND | Hypothetical protein |
| PA1535 | + | + | ND | ND | Probable acyl-CoA dehydrogenase |
| PA1555 fixP | − | + | ND | ND | Probable cytochrome c |
| PA1556 fixO | − | + | ND | ND | Probable cytochrome c oxidase subunit |
| PA1557 fixN | − | + | ND | ND | Probable cytochrome oxidase subunit (cbb3-type) |
| PA1558 | − | + | ND | ND | Hypothetical protein |
| PA1790 | + | + | ND | ND | Hypothetical protein |
| PA1892 | + | + | ND | + | Hypothetical protein |
| PA1893 | + | + | + | + | Hypothetical protein |
| PA1910 ufrA | + | + | ND | ND | Probable tonB-dependent receptor protein |
| PA2034 | + | + | ND | ND | Hypothetical protein |
| PA2095 | + | + | ND | ND | Hypothetical protein |
| PA2122 | + | + | ND | ND | Hypothetical protein |
| PA2383 | + | + | ND | ND | Probable transcriptional regulator |
| PA2384 | + | + | ND | ND | Hypothetical protein |
| PA2404 | + | + | ND | ND | Hypothetical protein |
| PA2413 | + | + | ND | ND | Probable class III aminotransferase |
| PA2508 catC | + | − | ND | ND | Muconolactone delta isomerase |
| PA2619 infA | − | + | ND | ND | Initiation factor |
| PA2687 pfeS | + | − | ND | ND | Two-component sensor |
| PA2831 | + | − | ND | ND | Hypothetical protein |
| PA2907 cobL | + | + | ND | ND | Precorrin-6y-dependent methyltransferase |
| PA3126 ibpA | + | + | ND | ND | Heat-shock protein |
| PA3535 eprS | + | − | + | − | Probable serine protease |
| PA4009 | + | − | ND | ND | Hypothetical protein |
| PA4182 | + | − | ND | ND | Hypothetical protein |
| PA4206mexH | + | − | + | + | Probable RND efflux membrane fusion protein precursor |
| PA4588gdhA | + | − | ND | ND | Glutamate dehydrogenase |
| PA4658 | − | − | ND | ND | Hypothetical protein |
| PA4810 fdnl | + | + | ND | ND | Nitrate-inducible formate dehydrogenase, γ subunit |
| PA4909 | + | + | ND | ND | Probable ATP-binding comp. of ABC transporter |
| PA4913 | + | + | ND | ND | Probable binding protein comp. of ABC transporter |
| PA4973 thiC | + | + | ND | ND | Thiamine biosynthesis protein |
| PA5188 | + | + | ND | ND | Probable 3-hydroxyacyl-CoA dehydrogenase |
| PA5380 | + | + | ND | ND | Probable transcriptional regulator |
| PA5460 | + | + | ND | ND | Hypothetical protein |
| PA5471 | + | + | ND | ND | Hypothetical protein |
| PA5510 | + | − | ND | ND | Probable transporter |

*Upregulation (+), Downregulation (−), 1. PI this study 2. PA01.
†Negative at 24 h, positive thereafter.
ND = No differential expression detected.
Gene ID in bold = Contiguous genes-putative operon.

TABLE 10
Genes differentially regulated in PI and simultaneously in the
Liverpool epidemic strains LES400 and LES431. The expression
profiles of the LES strains were compared to that of PA01.

| | | | | |
|---|---|---|---|---|
| | | Fold- | Fold- | |
| Gene | Fold-PI | LES400 | LES431 | Description |

All PI and Unique isolates

| | | | | |
|---|---|---|---|---|
| PA0364 | 2.6 | 5.4 | 4.6 | Probable oxidoreductase |
| PA1250 | 3.7 | ND | 16 | Alkaline proteinase inhibitor |
| aprI | | | | |
| PA2587 | 2.7 | ND | 5.7 | Probable FAD-dependent |
| pqsH | | | | monooxygenase |
| PA3535 | 1.8 | ND | 10 | Probable serine protease |
| PA3904 | 3.1 | ND | 9.9 | Hypothetical protein |
| PA4590 | 2.5 | ND | 27 | Protein activator |
| pra | | | | |
| PA4738 | 3.0 | ND | 19 | Hypothetical protein |
| PA4739 | 3.5 | ND | 26 | Hypothetical protein |
| PA5220 | 4.2 | ND | 26 | Hypothetical protein |
| PA5481 | 3.1 | ND | 38 | Hypothetical protein |

PI and Unique isolates from older patients

| | | | | |
|---|---|---|---|---|
| PA0364 | 5.2 | ND | 4.6 | Probable oxidoreductase |
| PA1001 | −5.1 | ND | 12.1 | Anthranilate synthase component I |
| phnA | | | | |
| PA1177 | −4.1 | ND | 5.8 | Periplasmic nitrate |
| napE | | | | reductase protein |
| PA1250 | 11.1 | ND | 16 | Alkaline proteinase inhibitor |
| aprI | | | | |
| PA1431 | 51.7 | −35 | 20 | Regulatory protein |
| rsaL | | | | |
| PA1893 | 2.2 | ND | 6.2 | Hypothetical protein |
| PA2007 | −9.5 | 15 | 16 | Maleylacetoacetate isomerase |
| maiA | | | | |
| PA2008 | −4.4 | 8.1 | 6.9 | Fumarylacetoacetase |
| fahA | | | | |
| PA2009 | −10.1 | 7.8 | −9.5 | Homogentisate 1,2-dioxygenase |
| hmgA | | | | |
| PA2513 | 54.1 | 18 | 50 | Anthranilate dioxygenase |
| antB | | | | small subunit |
| PA2587 | 5.0 | ND | 5.7 | Probable FAD-dependent |
| pqsH | | | | monooxygenase |
| PA2588 | 2.0 | ND | 26 | Probable transcriptional regulator |
| PA3535 | 2.6 | ND | 10 | Probable serine protease |
| PA3904 | 6.2 | ND | 9.9 | Hypothetical protein |
| PA3907 | 6.5 | ND | 7.4 | Hypothetical protein |
| PA3923 | 4.4 | −5.9 | 5.9 | Hypothetical protein |
| PA4206 | 3.2 | ND | 11 | Probable RND efflux |
| mexH | | | | membrane fusion protein |
| PA4498 | 2.4 | −14 | −3.1 | Probable metallopeptidase |
| PA4778 | 4.7 | ND | 6.4 | Probable transcriptional regulator |
| PA5061 | 1.6 | ND | 3.1 | Hypothetical protein |

ND No differential expression detected.
Gene ID in bold = Contiguous genes-putative operon.

1. A method for determining whether an opportunistic bacterium is infectious including assessing an opportunistic bacterium for the expression of a gene having a sequence that is the same as or homologous to the sequence of PA0729.

2. The method of claim 1, further including the step of assessing the bacterium for the expression of a gene having a sequence that is the same as the sequence of a gene selected from the group consisting of PA0632, PA0633, PA0636, PA0637, PA0638, and PA0639.

3. The method of claim 1, further including the step of assessing the bacterium for the expression of a gene having a sequence that is the same as the sequence of gene PA0986.

4. The method of claim 1, further including the step of assessing the bacterium for the expression of a gene having a sequence that is the same as the sequence of gene PA1617.

5. The method of claim 1, further including the step of assessing the bacterium for the expression of a gene having a sequence that is the same as the sequence of gene PA3067.

6. The method of claim 1, further including the step of assessing the bacterium for the expression of a gene having a sequence that is the same as the sequence of gene PA5429.

7. The method of claim 1, further including the step of assessing the bacterium for the expression of a gene having a sequence that is the same as the sequence of gene PA5471.

8. The method of claim 1 wherein the opportunistic bacterium is Psuedomonas aeruginosa.
